(19) United States
(12) Patent Application Publication
(10) Pub. No.: US 2023/0210852 A1
(43) Pub. Date: Jul. 6, 2023

(54) METHODS OF TREATING CANCER IN PATIENTS WITH AN ANOMALOUS KRAS GENE OR DELETIONS WITHIN CHROMOSOME 9

(71) Applicant: Syros Pharmaceuticals, Inc., Cambridge, MA (US)

(72) Inventors: John Graeme Hodgson, Boxborough, MA (US); Liv Helena Johannessen, Somerville, MA (US); Nisha Rajagopal, Boston, MA (US); Anthony D'ppolito, Acton, MA (US)

(21) Appl. No.: 17/928,529

(22) PCT Filed: May 28, 2021
(86) PCT No.: PCT/US2021/034979

§ 371 Date: Nov. 29, 2022

Related U.S. Application Data
(60) Provisional application No. 63/032,060, filed on May 29, 2020.

(51) Int. Cl. A61K 31/506; A61K 31/337; A61K 31/519; A61P 35/00

(52) U.S. Cl. CPC A61K 31/506; A61K 31/337; A61K 31/519; A61P 35/00

(57) ABSTRACT

The present invention relates to methods of identifying patients suffering from various types of cancer who are more likely to respond to treatment with a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof), either when administered or used alone or in combination with a second therapeutic agent (e.g., another anti-cancer therapy). Patients are identified based on one or more features (e.g., gene copy number or expression level) of certain biomarkers (e.g., KRAS and/or the chromosomal band 9q34).

### CROSS-REFERENCE TO RELATED APPLICATIONS

This application claims the benefit of the filing date of U.S. Provisional Application No. 63/032,060, filed May 29, 2020, the content of which is hereby incorporated by reference herein in its entirety.

### BACKGROUND OF THE INVENTION

The long evolution of healthcare has reached a point in time where the promise of biomarker analysis is beginning to be realized. When physicians can stratify patients, even those who share many similar physiological traits and exhibit common symptoms of a given disease, into more specific groups, they can better tailor treatment and optimize the outcome for each patient. However, it is challenging to develop molecular diagnostics, and few are commercially avaiable.

### SUMMARY OF THE INVENTION

The present invention features, inter alia, diagnostic methods for identifying cancer patients for treatment with a CDK7 inhibitor and, particularly, with a non-covalent and/or selective CDK7 inhibitor, as described herein (i.e., diagnostic methods for selecting a patient for treatment as described herein). The invention also features methods for treating identified patients with such an inhibitor, either alone or in combination with one or more additional therapeutic agents (i.e., a second anti-cancer agent), as described further below The diagnostic methods include a step of identifying a patient suffering from a cancer (e.g., a colorectal, lung, or pancreatic cancer expressing a KRAS or NRAS biomarker, a cancer affecting an organ of the reproductive tract (e.g., a breast, ovarian, or uterine cancer) and expressing a KRAS or NRAS biomarker; and/or a cancer of an organ of the reproductive organ (e.g., the uterus or ovary), bile duct, the skin, bladder, liver, lung, kidney, or bone expressing a 9q34 biomarker as described herein) that is likely to respond well to treatment with a CDK7 inhibitor such as THZ1, THZ2, SY-1365, YKL-5-124, ICEC0942, LY3405105, LDC4297, BS-181, alvocidib, seliciclib, SNS-32, or a compound of structural Formula (I), (Ia), a species thereof, or a pharmaceutically acceptable salt thereof (i.e., a pharmaceutically acceptable salt of any of the foregoing compounds). The treatment methods include a step of administering a therapeutically effective amount of such a CDK7 inhibitor to an identified patient, whose response can be, for example, significant tumor growth inhibition (TGI; e.g., more than about 50, 60, 70, 80, or 90% TGI), preferably with continued tumor suppression even after cessation of treatment and/or improved likelihood of progression-free or overall survival. Thus, the present invention encompasses methods in which a patient is only diagnosed as being a good candidate for treatment (i.e., identified for treatment as described herein), methods in which a patient who has been determined to be a good candidate for treatment is treated, and methods requiring that a patient be both diagnosed and treated as described herein.

The methods that require identifying a patient for treatment include a step of analyzing one or more of the biomarkers described herein in a biological sample obtained from the patient by determining, having determined, or receiving information concerning the state of the biomarker (as described further herein). In various embodiments, the state is assessed based on the presence, absence (e.g., a genetic deletion), location (e.g., chromosomal translocation), or copy number (e.g., duplication) of a biomarker gene or isoform thereof in wild type or mutant form, the inclusion of epigenetic modifications, the association of a biomarker gene with a super-enhancer (SE) or a SE of a certain strength, ordinal rank, or prevalence rank, the level of expression of the biomarker gene (as evidenced by, for example, the level of expression of a primary RNA transcript or a cDNA reverse-transcribed therefrom, and/or the level of expression or activity of the protein encoded by the biomarker gene. These features of a given biomarker are discussed further below and their analysis can be incorporated in any of the methods described above. Moreover, the state of a biomarker, determined by analyzing any one or more of the features just listed (e.g., the presence of a mutation or a deletion), can be assessed for any of the present biomarkers (e.g., KRAS, KRAS or 9q34), alone or in combination with each other or another biomarker. As described further below, CDK9, MED22, and NUP214 reside on chromosomal band 9q34 and can corroborate or serve as surrogates for its deletion in any of the present methods; our data show lower expression of these genes in 9q34-deleted colorectal cancer (CRC) PDX (patient-derived xenograft) models (such models are known in the art and reviewed by, for example, Koga and Ochiai, Cells 8(5):418, 2019; doi: 10.3390/cells8050418). Thus, in one embodiment of the present methods, regardless of the precise method carried out (e.g., whether diagnostic or therapeutic, either of which may be carried out with any of the sample types, analytical reagents, and methods described herein or known in the art); or regardless of the context in which the biomarker is being assessed (e.g., regardless of the patient’s cancer type) a biological sample comprising cancer cells from a patient is analyzed for KRAS (e.g., a KRAS-activating mutation), NRAS, and/or a complete or partial deletion of 9q34 as evidenced by, for example, reduced expression of CDK9, MED22, and/or NUP214. The state of a given biomarker (e.g., its sequence, including the specific mutations described herein), copy number, associated enhancer, expression level, or activity) may be equal to or above a pre-determined threshold level or cutoff or equal to or below a pre-determined threshold level or cutoff, as described further below. In the methods of the present invention, one can analyze KRAS or NRAS, RNA transcribed therefrom, or a protein encoded thereby (K-ras or N-ras, respectively) as described herein and/or chromosomal band 9q34, also as described herein, together with one or more of the following, additional biomarkers: BCL2L1, BRAF, DIS3 (for, e.g., amplification-dependent overexpression), WNT, chromosomal band 1p36 (for deletion, for example), msi (microsatellite instability), 8q (for amplification or gain of function), and 20q (for amplification or gain of function). In case of doubt, the additional biomarkers can be analyzed by assessing the gene of interest (for, e.g., an activating or deactivating mutation or association with a super-enhancer), an RNA encoded thereby (for, e.g., level of expression), or a protein translated therefrom (for, e.g., its level of activity), any of which can be assessed relative to a reference standard. In some embodiments, and as described further below, BCL2L1 and genes located within the biomarker/chromosomal bands 8q and 20q are more highly expressed in biological samples of patients less likely to respond to treatment with a CDK7 inhibitor (e.g., THZ1, THZ2, SY-1365, YKL-5-124, ICEC0942, LY3405105, LDC4297, BS-181, alvocidib, seliciclib, SNS-32, a compound of structural Formula (I), (Ia), a species thereof, or a pharmaceutically acceptable salt of any of the foregoing). For example, our data have shown that genes located on 8q are more highly expressed in animal models of human tumors that do not respond well to treatment with Compound 101; 8q13 and other 8q gains were associated with a weaker response to Compound 101 in a PDX model of CRC. BCL2L1 expression is also higher in PDX models with weaker responses to Compound 101. Regarding DIS3, we believe its amplification creates a transcriptional liability that is synthetically lethal with CDK7 inhibition. Exemplary combinations of useful biomarkers are illustrated in FIG. 5. For example, in addition to analyzing the state of a RAS gene (e.g., KRAS or NRAS or the RNA or protein encoded thereby) and/or 9q34 in a biological sample, one can analyze p53 as an additional biomarker. Alternatively, or in addition, one can analyze DIS3 (e.g., for amplification). Alternatively, or in addition to analyzing p53 and/or DIS3, one can analyze 20q (e.g., for gain of 20q11, 20q12, or 20q13). Alternatively, or in addition to analyzing p53, one can analyze 8q13 (e.g., for a gain of function). In various embodiments, the diagnostic methods comprise analysis of KRAS alone, NRAS alone, and/or 9q34 and: p53; p53 and DIS3; p53, DIS3, and 20q; p53 and 20q, p53 and 8q13; 20q and 8q13. In other embodiments, the methods encompass analysis of 9q34 and BRAF. Alternatively, or in addition, one can analyze 9q34 and p53 (e.g., 9q34, BRAF and p53). Alternatively, or in addition, one can analyze 20q (e.g., 20q11, 20q12, or 20q13 gain) (e.g., 9q34, BRAF, p53, and 20q). Other useful combinations will be evident from FIG. 5.

In embodiments of the methods described above, identifying a patient can be carried out by determining, having determined, and/or receiving information that KRAS or the protein it encodes is mutant or expressed at a level above a pre-determined threshold level, which mutation or expression level may lead to enhanced KRAS activity (e.g., a patient can be identified when a non-binary parameter is 20-80% different from (e.g., above) a reference standard or 1.5-5-fold different from (e.g., above) a reference standard, with the same threshold levels being applied to other biomarkers described herein). As noted, a KRAS mutation can be an activating mutation (e.g., a mutation encoding an amino acid substitution at position 12 (e.g., G12V, G12D, G12C, G12S, or G12A)) or 13. As noted, alternatively or in addition, one can analyze the chromosomal region designated 9q34 or the proteins encoded by one or more of the genes known to reside therein, by determining, having determined, and/or receiving information that the state of such biomarker is below a pre-determined threshold level. For example, 9q34 can be wholly or partially deleted.

Aliases, chromosomal locations, splice variants, and homologs of the genes and proteins described herein as biomarkers, in Homo sapiens and other species, are known in the art.

The treatment methods of the invention and corresponding “uses” include administering, or the use of, a CDK7 inhibitor, such as THZ1 (Kwiatkowski et al., Nature 511(7511):616-620, 2014; see also Li et al., Chronic Diseases and Translational Medicine 5:155-169, 2019), THZ2 (Wang et al., Cell 163(1):174-186, 2015), SY-1365 (Hu et al., Cancer Res. 79:3479-3491, 2019; WO 2015/154039; and U.S. Publication No. 2017-0183355, which is incorporated by reference herein in its entirety) YKL-5-124 (Olson et al., Cell Chemical Biology 26:792-803, 2019), ICEC0942 (also known as CT7001; Patel et al., Molecular Cancer Therapeutics 17(6):1156-1166, 2018; Hazel et al., ChemMedChem. 12(5):372-380, 2017; see also WO 2019/057825), LY3405105 (Coates et al., “Compounds useful for inhibiting CDK7. United States: Eli Lilly and company. IN, US: Indianapolis; WO 2019/099298), LDC4297 (Kelso et al. Molecular and Cellular Biology, 34(19):3675-3688), BS-181 (Ali et al. Cancer Research, 69(15):6208-6215; Wang et al. Drug Design, Development and Therapy, 10:1181-1189), alvocidib (a non-selective inhibitor; Kaur et al. J of the Nat Can Inst., 84(22):1736-1740; Losiewicz et al. Biochemical and Biophysical Research Communications, 201(2):589-595; Carlson et al., Cancer Research, 56(13) 2973-2978; and Chen et al. Blood, 106(7):251)-2519), seliciclib (also known as CYC202; Meijer et al. Eur. J. Biochem., 243(1-2):527-536; Whitttaker et al. Cancer Research, 64(1):262-272; McClue et al. Int. J. Cancer 102(5):463-468), SNS-032 (Nuwayhid et al. Proc. Am. Assoc. Cancer Res., 47:491, 2006) or a compound of Formula (I) (WO 2020/093011; see also U.S. Pat. No. 10,738,067, which is hereby incorporated by reference in its entirety), any of which may be included in a pharmaceutically acceptable composition and administered by a route and regimen described further herein or known in the art for that particular inhibitor, to a patient identified as described herein (see the diagnostic methods described above and elsewhere herein). In some embodiments, the CDK7 inhibitor is selective for CDK7 (e.g., THZ1. THZ2, SY-1365, YKL-5-124, ICEC0942, LY3405105, LDC4297, BS-181 or a compound of structural Formula (I) or (Ia)), and in other embodiments, the CDK7 inhibitor is non-selective (e.g., alvocidib). More specifically, a compound useful in the present methods has a structural formula shown in FIG. 7 or conforms to Formula (I):

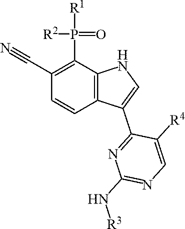

or is a pharmaceutically acceptable salt thereof (i.e., of any of the foregoing), wherein R¹ is methyl or ethyl; R² is methyl or ethyl; R³ is 5-methylpiperidin-3-yl, 5,5-dimethylpiperidin-3-yl, 6-methylpiperdin-3-yl, or 6,6-dimethylpiperidin-3-yl; and R⁴ is —CF₃ or chloro. In certain embodiments, the compound has structural Formula (Ia):

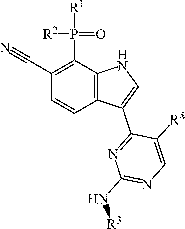

(Ia), wherein R³ is

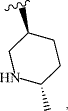

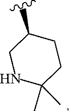

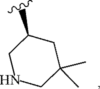

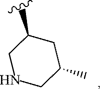

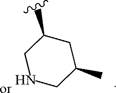

For example, the compound can be:

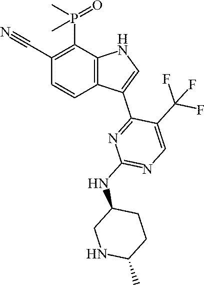 [SMILES: C[C@H]1CC[C@H](Nc2ncc(C(F)(F)F)c(-c3c[nH]c4c(P(C)(C)=O)c(C#N)ccc34)n2)CN1]

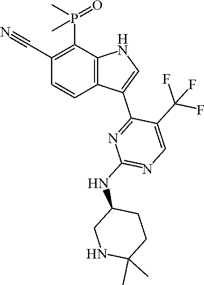 [SMILES: CC1(C)CC[C@H](Nc2ncc(C(F)(F)F)c(-c3c[nH]c4c(P(C)(C)=O)c(C#N)ccc34)n2)CN1]

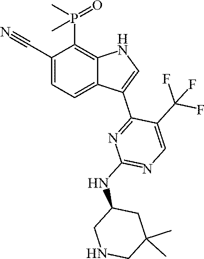 [SMILES: CC1(C)CNC[C@@H](Nc2ncc(C(F)(F)F)c(-c3c[nH]c4c(P(C)(C)=O)c(C#N)ccc34)n2)C1]

In one embodiment, the compound is

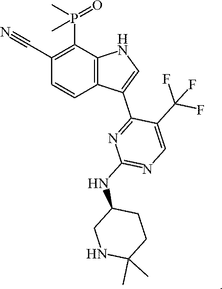

A CDK7 inhibitor useful in the present methods can be any compound described herein, including those shown in FIG. 7 and a compound of Formula (I), (Ia), or a species thereof, and can be in the form of a pharmaceutically acceptable salt as described further herein or known in the art. Any of these pharmaceutically acceptable salts can be contained within a pharmaceutically acceptable composition as described further herein or known in the art (e.g., formulated for oral or parenteral (e.g., intravenous) administration).

For the present diagnostic methods, one can determine that a patient’s cancer is more likely to respond to treatment with a CDK7 inhibitor (e.g., THZ1, THZ2, SY-1365, YKL-5-124, ICEC0942, LY3405105, LDC4297, BS-181, alvocidib, seliciclib, SNS-32, a compound of structural Formula (I), (Ia), a species thereof, or a pharmaceutically acceptable salt of any of the foregoing) when a biological sample obtained from the patient (e.g., a sample of blood (e.g., comprising circulating tumor DNA) or biopsied tissue) is determined to include a biomarker described herein (e.g., KRAS or 9q34) with any one or more of the anomalies (e.g., mutations or deletions) described herein. For example, to determine whether a patient’s cancer is associated with a KRAS biomarker (i.e., is “KRAS-positive”), the methods can include a step of analyzing, from the biological sample, the sequence of KRAS for the presence of a mutation (e.g., a mutation that increases the activity of the encoded protein); a step of analyzing the genome of the sampled cancer cells to detect translocations, epigenetic modifications, or amplifications of KRAS that, for example, lead to its overexpression and/or overactivity; a step of assessing the level of a KRAS RNA transcript (e.g., KRAS eRNA, a primary RNA transcript, or mRNA) and/or a step of analyzing the encoded protein for anomalous (e.g., increased) expression levels or activity. In each case, the biomarker information can be compared to a reference standard (e.g., to sequences or to expression or activity levels of the same biomarker in a population of patients who do not have cancer or do not have the type of cancer from which the patient is suffering). Similarly, and for example, to determine whether a patient’s cancer is associated with a 9q34 deletion and is therefore more likely to respond to treatment with a CDK7 inhibitor (e.g., THZ1, THZ2, SY-1365, YKL-5-124, ICEC0942, LY3405105, LDC4297, BS-181, alvocidib, seliciclib, SNS-32, or a compound of structural Formula (I), (Ia), a species thereof, or a pharmaceutically acceptable salt thereof), the methods of identifying, diagnosing, and/or treating a patient can include a step of analyzing the genome of cells within the biological sample for a deletion of all or a part (e.g., a significant part) of the chromosomal band 9q34 or determining whether levels of expression of at least two genes located within that band, or the proteins they encode, are below the level of a reference standard (e.g., below an expression level in a population of patients who do not have cancer or do not have the type of cancer from which the “9q34-positive” patient is suffering). In case of doubt, analogous steps can be carried out for any of the additional biomarkers described herein.

For the present methods of treatment and corresponding uses of a CDK7 inhibitor (e.g., THZ1, THZ2, SY-1365, YKL-5-124, ICEC0942, LY3405105, LDC4297, BS-181, alvocidib, seliciclib, SNS-32, or a compound of structural Formula (I), (Ia), a species thereof, or a pharmaceutically acceptable salt thereof), a therapeutically effective amount of such an inhibitor can be administered to a patient determined to have a KRAS-positive and/or 9q34-positive cancer selected from any cancer type, including those described below as amenable to treatment and, optionally, further analyzed for any one or more of the secondary biomarkers described herein. In one embodiment, the cancer expresses a KRAS biomarker (i.e., is determined to be KRAS-positive) and is a colorectal, lung (e.g., non-small cell lung cancer (NSCLC)), or pancreatic cancer (e.g., pancreatic ductal adenocarcinoma (PDAC)). In another embodiment, the KRAS-positive cancer is a breast (e.g., a hormone receptor-positive (HR+) or triple-negative breast cancer (TNBC)) or ovarian cancer (e.g., high-grade serous ovarian cancer (HGSOC)). In one embodiment, the cancer expresses a 9q34 biomarker (i.e., is determined to be 9q34-positive) and arises within a reproductive organ (e.g., the cancer can be a uterine cancer (e.g., uterine carcinosarcoma (UCS) or uterine corpus endometrial carcinoma (UCEC)) or ovarian cancer (e.g., ovarian serous cystadenocarcinoma)), bile duct (i.e., is a cholangiocarcinoma), the skin (e.g., a melanoma), bladder, liver, lung (e.g., mesothelioma), kidney (e.g., chromophobe renal cell carcinoma (KICH)), or bone (e.g., a sarcoma).

For the present methods of treatment and corresponding uses of a CDK7 inhibitor (e.g., THZ1, THZ2, SY-1365, YKL-5-124, ICEC0942, LY3405105, LDC4297, BS-181, alvocidib, seliciclib, SNS-32, or a compound of structural Formula (I), (Ia), a species thereof, or a pharmaceutically acceptable salt thereof), a therapeutically effective amount of such an inhibitor can be administered to a patient identified as described herein in combination with at least one additional therapeutic agent, as discussed further below. For example, a CDK7 inhibitor, including one such as a non-covalent CDK7 inhibitor represented by structural Formula (I), (Ia), a species thereof, or a pharmaceutically acceptable salt thereof, can be administered together with or subsequent to a standard-of-care chemotherapeutic agent such as alpelisib, binimetinib, trametinib, or gemcitabine. In specific embodiments, a patient having CRC can be assessed as described herein (i.e., a biological sample obtained from the patient can be determined to express a biomarker or plurality thereof, as described herein (e.g., KRAS and/or 9q34, with BRAF as a secondary biomarker)) and treated with a CDK7 inhibitor (e.g., THZ1, THZ2, SY-1365, YKL-5-124, ICEC0942, LY3405105, LDC4297, BS-181, alvocidib, seliciclib, SNS-32, or a compound of structural Formula (I), (Ia), a species thereof, or a pharmaceutically acceptable salt thereof) and one or more of binimetinib, encoratenib, and trametinib; a patient having Ewing’s sarcoma can be treated with a CDK7 inhibitor (e.g., as just referenced) and one or more of irinotecan, temozolimide, and a PARP inhibitor (e.g., olaparib); a patient having a pancreatic cancer (e.g., PDAC, determined to be associated with, e.g., a KRAS biomarker) can be treated with a CDK7 inhibitor (e.g., as just referenced) and one or more of binimetinib, trametinib, and gemcitabine; a patient having a breast cancer (e.g., TNBC) or ovarian cancer (e.g., HGSOC) can be treated with a CDK7 inhibitor (e.g., as just referenced) and one or more of gemcitabine, a platinum-based therapeutic agent (e.g., carboplatin), topotecan, and doxorubicin; a patient having a small cell lung cancer (SCLC) can be treated with a CDK7 inhibitor (e.g., as just referenced) and one or more of gemcitabine, carbolatinum, topotecan, a taxane (e.g., docetaxol), and temozolimide; or a patient having a NSCLC determined to be associated with, e.g., a KRAS biomarker, can be treated with a CDK7 inhibitor (e.g., as just referenced) and one or more of binimetinib, trametinib, and a taxane (e.g., docetaxol).

Another aspect of the present invention features methods of treating a patient as described immediately above regardless of biomarker status.

### BRIEF DESCRIPTION OF THE DRAWINGS

FIG. 1 is a graph depicting body weight changes at the end of treatment in vehicle- and Compound 101-treated PDX mouse models, as described in Example 1. Each point represents the average body weight loss of an independent CRC PDX model (n=3 mice per model). The heavier gray horizontal bars represent the average body weight change across all 30 models.

FIG. 2 is a graph illustrating the anti-tumor activity of Compound 101 at 6 mg/kg in the 30 CRC PDX models described in Example 1. Each circle represents the %TGI at EoT (end of treatment). The dashed horizontal lines represent 50% and 90% TGI cut offs.

FIG. 3 is a graph illustrating the growth rate (GR) inhibition of cells of the KRAS mutant cell line SW480 upon exposure in cell culture to varying amounts of Compound 101 (downward triangles), trametinib (diamonds) and binimetinib (circles).

FIG. 4 is a pair of isobolograms illustrating the synergistic effect of Compound 101 and trametinib when administered in combination to the KRAS mutant cell line SW480 (each isobologram represents the result of one experiment).

FIG. 5 is an illustration of our data analysis mapping seven features (BRAF_mut; 9q34_loss, DIS3_amp, TP53_mut, msi, 8q13_gain, and 20q11_20q12_20q13_gain) according to their presence (darker gray) or absence (lighter gray) in the 30 PDX models indicated along the base of the figure, along with the response in each model to Compound 101 (first two rows) and subtype (third row). Responses of ≥ 75% (+) are indicated in black (row 1, columns 1-10) and negative responses (-) are indicated in white (row 1, columns 11-24). TGI appears grey-scaled in row 2, with the PDX models demonstrating greater TGI positioned in row 2, toward column 1. The subtype of each model is clear in FIG. 5, KRAS mutants are in row 3, columns 2, 8, 11-12, 16-19, and 21, BRAF mutant models are in row 3, columns 1, 3-4, 6-7, 9, 13, and 15, and WT mutant models in row 3, columns 5, 10, 14, 20, 22-24.

FIG. 6 is a panel of line graphs illustrating tumor volume in vehicle-treated ((light gray circles) and Compound 101-treated (darker gray squares) in two BRAF mutant- and one KRAS-mutant PDX model. Complete tumor regression was observed and all three models carried 9q34 heterorygous deletions.

FIG. 7 is an illustration of CDK7 inhibitors described herein for use in a kit or any one or more of the present methods (e.g., diagnostic and/or therapeutic methods in which the CDK7 inhibitor is administered or used alone or in combination with a second anti-cancer agent).

FIG. 8 is a table summarizing the anti-tumor activity of Compound 101 provided at a dose of 6 mg/kg QD in each of the eight PDAC PDX models described in Example 4.

FIGS. 9A-9B depict the enhanced effect of a combination of Compound 101 and gemcitabine on the growth of PANC-1 cells (a KRAS mutant PDAC cell line). FIG. 9A shows the dose response curves and an isobologram indicating a synergistic effect of Compound 101 and gemcitabine on the growth of the PANC-1 cells. FIG. 9B is a panel of photographs showing the results of colony formation assays of the PANC-1 cells treated as indicated with Compound 101 (at 0, 0.5, and 5 nM) and gemcitabine (at 0 or 5 nM). The visualization of colony formation was aided by crystal violet staining at Day 6.

FIG. 10 is a line graph depicting tumor volume (mm³) over time (days) in a PDAC CDX model PANC-1. Black lines with circles represent vehicle-treated animals. Gray lines with inverted triangles represent gemcitabine-treated animals. Darker gray lines with squares represent Compound 101-treated animals. Black dashed line with diamonds represents a combination therapy with Compound 101 and gemcitabine.

FIGS. 11A-11C depict the enhanced effect of a combination of Compound 101 and docetaxel on the growth of A549 cells (a KRAS mutant lung cancer cell line). FIG. 11A shows the dose response curves and an isobologram indicating a synergistic effect of Compound 101 and docetaxel on the growth of the A549 cells. FIG. 11B is a panel of photographs showing the results of colony formation assays of the A549 cells treated as indicated with Compound 101 (at 0, 1, and 5 nM) and docetaxel (at 0 and 0.4 nM). The visualization of colony formation was aided by crystal violet staining at Day 6. FIG. 11C is a bar graph depicting the effect on cellular growth phases and the enhanced effect of Compound 101 and docetaxel when administered in combination to the KRAS mutant cell line A549.

FIGS. 12A and 12B are line graphs depicting the response of A549 cells (a KRAS mutant cell line model of NSCLC; FIG. 12A) and ST2972 cells (a KRAS mutant cell line model of NSCLC; FIG. 12B) to various daily dosages of Compound 101, docetaxel, and the combination of Compound 101 and docetaxel.

FIG. 13 is a line graph depicting tumor volume (mm³) over time (days) in mice injected with PANC-1 cells and treated with various daily dosages of Compound 101, trametinib, the combination of compound 101 and trametinib, BI-3406, and the combination of Compound 101 and BI-3406. See Example 9.

FIG. 14 is a line graph depicting tumor volume (mm³) over time (days) to illustrate response to various daily dosages of compound 101, BI-3406 and the combination of compound 101 and B1-3406.

FIG. 15 shows the growth rate curves for 24 breast cancer and ovarian cancer cell lines treated with compound 101 in vitro. Cell lines that are 9q34 deleted are colored black. Note the deeper response of the 9q34 lines (GR max).

FIG. 16 shows the comparison of the GR max of 24 breast cancer and ovarian cancer cell lines. GR max: growth rate at the maximum concentration. Note the 9q34 het deleted lines have a statistically significant lower GR max

### DETAILED DESCRIPTION

Despite the efficacy of CDK7 inhibitors, including compounds of Formula (I), we believe there is a benefit to identifying and treating patients that have certain genetic signatures (i.e., biomarkers in a particular state, as described herein). Moreover, the efficacy of CDK7 inhibitors (e.g., THZ1, THZ2, SY-1365, YKL-5-124, ICEC0942, LY3405105, LDC4297, BS-181, alvocidib, seliciclib, SNS-32, a compound of structural Formula (I), (Ia), a species thereof, or a pharmaceutically acceptable salt of any of the foregoing) may be enhanced when combined with other anti-cancer therapies in patients identified as described herein.

The following definitions apply to the compositions, methods, and uses described herein. Moreover, the definitions apply to linguistic and grammatical variants of the defined terms (e.g., the singular and plural forms of a term), and some linguistic variants are particularly mentioned below (e.g., “administration” and “administering”). The chemical elements are identified in accordance with the Periodic Table of the Elements, CAS version, Handbook of Chemistry and Physics, 75^{th} Ed. Additionally, general principles of organic chemistry are well established and one of ordinary skill in the art can consult Organic Chemistry by Thomas Sorrell, University Science Books, Sausalito, 1999, Smith and March, March’s Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The term “about,” when used in reference to a value, signifies any value or range of values that is plus-or-minus 10% of the stated value (e.g., within plus-or-minus 1%, 2%, 3%, 4%, 5%, 6%, 7%, 8%, 9% or 10% of the stated value). For example, a dose of about 10 mg means any dose as low as 10% less than 10 mg (9 mg), any dose as high as 10% more than 10 mg (11 mg), and any dose or dosage range therebetween (e.g., 9-11 mg; 9.1-10.9 mg; 9.2-10.8 mg; and so on). As another example, a prevalence rank in a population of about 80% means a prevalence rank of 72-88% (e.g., 79.2-80.8%). In case of doubt, “about X” can be “X” (e.g., about 80% can be 80%). Where a stated value cannot be exceeded (e.g., 100%), “about” signifies any value or range of values that is up to and including 10% less than the stated value (e.g., a purity of about 100% means 90%-100% pure (e.g., 95%-100% pure, 96%-100% pure, 97%-100% pure etc... )). In the event an instrument or technique measuring a value has a margin of error greater than 10%, a given value will be about the same as a stated value when they are both within the margin of error for that instrument or technique.

The term “administration” and variants thereof, such as “administering,” refer to the administration of a compound described herein (e.g., THZ1, THZ2, SY-1365, YKL-5-124, ICEC0942, LY3405105, LDC4297, BS-181, alvocidib, seliciclib, SNS-32, and pharmaceutically acceptable salts thereof, or a compound of structural Formula (I), (Ia), a species thereof, and pharmaceutically acceptable salts thereof) or an additional/second agent), or a composition containing the compound to a subject (e.g., a human patient) or system (e.g., a cell- or tissue-based system that is maintained ex vivo): as a result of the administration, the compound or composition containing the compound (e.g., a pharmaceutical composition) is introduced to the subject or system. In addition to selective and non-selective CDK7 inhibitors and second agents useful in combination therapies, items used as positive controls, negative controls, and placebos, any of which can also be a compound, can also be “administered.” One of ordinary skill in the art will be aware of a variety of routes that can, in appropriate circumstances, be utilized for administration to a subject or system. For example, the route of administration can be oral (i.e., by swallowing a pharmaceutical composition) or may be parenteral. More specifically, the route of administration can be bronchial (e.g., by bronchial instillation), by mouth (i.e., oral), dermal (which may be or comprise topical application to the dermis or intradermal, interdermal, or transdermal administration), intragastric or enteral (i.e., directly to the stomach or intestine, respectively), intramedullary, intramuscular, intranasal, intraperitoneal, intrathecal, intratumoral, intravenous (or intra-arterial), intraventricular, by application to or injection into a specific organ (e.g., intrahepatic), mucosal (e.g., buccal, rectal, sublingual, or vaginal), subcutaneous, tracheal (e.g., by intratracheal instillation), or ocular (e.g., topical, subconjunctival, or intravitreal). Administration can involve intermittent dosing (i.e., doses separated by various times) and/or periodic dosing (i.e., doses separated by a common period of time (e.g., every so many hours, daily (e.g., once daily oral dosing), weekly, twice per week, etc.)). In other embodiments, administration may involve continuous dosing (e.g., perfusion) for a selected time (e.g., about 1-2 hours)

Two events, two entities, or an event and an entity are “associated” with one another if one or more features of the first (e.g., its presence, level and/or form) are correlated with a feature of the second. For example, a first entity (e.g., an enzyme (e.g., CDK7)), gene expression profile, genetic signature (i.e., a single or combined group of genes in a cell with a uniquely characteristic pattern of gene expression), metabolite, or event (e.g., myeloid infiltration)) is associated with an event (e.g., the onset or progression of a particular disease), if its presence, level and/or form correlates with the incidence of, severity of, and/or susceptibility to the disease (e.g., a cancer disclosed herein). The biomarkers described herein are associated with an identified cancer in a patient in the manner described herein (e.g., by virtue of their sequence, copy number, level of expression, etc.). Associations are typically assessed across a relevant population. Two or more entities are physically “associated” with one another if they interact, directly or indirectly, so that they are and/or remain in physical proximity with one another in a given circumstance (e.g., within a cell maintained under physiological conditions (e.g., within cell culture) or within a pharmaceutical composition). Entities that are physically associated with one another can be covalently linked to one another or non-covalently associated by, for example, hydrogen bonds, van der Waals forces, hydrophobic interactions, magnetism, or combinations thereof. A compound of Formula (I), (Ia), a species thereof, or a pharmaceutically acceptable salt thereof can be non-covalently associated with CDK7.

The term “biological sample” refers to a sample obtained or derived from a biological source of interest (e.g., a tissue or organism (e.g., an animal or human patient) or cell culture). For example, a biological sample can be a sample obtained from an individual (e.g., a patient or an animal model) suffering from a disease (or, in the case of an animal model, a simulation of that disease in a human patient) to be diagnosed and/or treated by the methods of this invention or from an individual serving in the capacity of a reference or control (or whose sample contributes to a reference standard or control population). The biological sample can contain a biological cell, tissue or fluid or any combination thereof. For example, a biological sample can be or can include ascites; blood, blood cells; a bodily fluid, any of which may include or exclude cells (e.g., tumor cells (e.g., circulating tumor cells (CTCs) found in at least blood or lymph vessels)); bone marrow or a component thereof (e.g., hematopoietic cells, marrow adipose tissue, or stromal cells); cerebrospinal fluid (CSF); feces; flexural fluid; free-floating nucleic acids (e.g., circulating tumor DNA), gynecological fluids; immune infiltrates; lymph; peritoneal fluid; plasma; saliva; sputum; surgically-obtained specimens; tissue scraped or swabbed from the skin or a mucus membrane (e.g., in the nose, mouth, or vagina); tissue or fine needle biopsy samples; urine; washings or lavages such as a ductal lavage or broncheoalveolar lavage; or other body fluids, tissues, secretions, and/or excretions. Samples of, or samples obtained from, a bodily fluid (e.g., blood, CSF, lymph, plasma, or urine) may include tumor cells (e.g., CTCs) and/or free-floating or cell-free nucleic acids of the tumor. Cells (e.g., cancer cells) within the sample may have been obtained from an individual patient for whom a treatment is intended. Samples used in the form in which they were obtained may be referred to as “primary” samples, and samples that have been further manipulated (e.g., by removing one or more components of the sample) may be referred to as “secondary” or “processed” samples. Such processed samples may contain or be enriched for a particular cell type (e.g., a CDK7-expressing cell, which may be a tumor cell), cellular component (e.g., a membrane fraction), or cellular material (e.g., one or more cellular proteins, including CDK7, DNA, or RNA (e.g., mRNA), which may encode CDK7 and may be subjected to amplification). As used herein, the term “biomarker” refers to an entity whose state correlates with a particular biological event so that it is considered to be a “marker” for that event (e.g., the presence of a particular cancer and its susceptibility to THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof). A biomarker can be analyzed at the nucleic acid or protein level; at the nucleic acid level, one can analyze the presence (e.g., copy number alterations (CNAs)), absence, sequence, or chromosomal location of a gene in wild type or mutant form, epigenetic alterations (in, e.g., methylation), its association with a super-enhancer, and/or its level of expression (as evidenced, for example, by the level of a primary RNA transcript). For example, where the biomarker is KRAS, one can assess a biological sample obtained from a patient for the presence of mutations in the KRAS gene, and those mutations may be the same as manifest in the established models ST865, ST1660B, ST230, ST046, ST491C, ST1354, ST1192, ST094, ST238, and ST042 (for example). Where the biomarker is BRAF, one can assess a biological sample obtained from a patient for the presence of mutations in the BRAF gene, and those mutations may be the same as those manifest in the established models ST1207, ST428, ST540, ST2161, ST2148, ST1053, T1975, ST1163, and ST1419 (for example). At the protein level, one can analyze the level of expression and/or activity of a protein encoded by a biomarker gene. A biomarker may indicate a therapeutic outcome or likelihood (e.g., increased likelihood) thereof (e.g., responsiveness to a CDK7 inhibitor described herein). Thus, biomarkers can be predictive or prognostic and are therefore useful in methods of identifying/diagnosing and/or treating a patient (e.g., a selected patient) as described herein.

The term “cancer” refers to a disease in which biological cells exhibit an aberrant growth phenotype characterized by loss of control of cell proliferation to an extent that will be detrimental to a patient having the disease. A cancer can be classified by the type of tissue in which it originated (histological type) and/or by the primary site in the body in which the cancer first developed. Based on histological type, cancers are generally grouped into six major categories: carcinomas, sarcomas; myelomas; leukemias; lymphomas; and mixed types. A cancer analyzed and/or treated as described herein may be of any one of these types and may comprise cells that are precancerous (e.g., benign), malignant, pre-metastatic, metastatic, and/or non-metastatic. A patient who has a malignancy or malignant lesion has a cancer. The present disclosure specifically identifies certain cancers to which its teachings may be particularly relevant, and one or more of these cancers may be characterized by a solid tumor or by a hematologic tumor, which may also be known as a blood cancer (e.g., of a type described herein). Although not all cancers manifest as solid tumors, we may use the terms “cancer cell” and “tumor cell” interchangeably to refer to any malignant cell. More specific cancer types (e.g., breast, CRC, lung, etc.) amenable to the methods described herein are discussed further below.

The term “combination therapy” refers to those situations in which a subject is exposed to two or more therapeutic regimens (e.g., two or more therapeutic agents) to treat a single disease (e.g., a cancer). The two or more regimens/agents may be administered simultaneously or sequentially. When administered simultaneously, a dose of the first agent and a dose of the second agent are administered at about the same time, such that both agents exert an effect on the patient at the same time or, if the first agent is faster- or slower-acting than the second agent, during an overlapping period of time. When administered sequentially, the doses of the first and second agents are separated in time, such that they may or may not exert an effect on the patient at the same time. For example, the first and second agents may be given within the same hour or same day, in which the first agent would likely still be active when the second is administered. Alternatively, a much longer period of time may elapse between administration of the first and second agents, such that the first agent is no longer active when the second is administered (e.g., all doses of a first regimen are administered prior to administration of any dose(s) of a second regimen by the same or a different route of administration, as may occur in treating a refractory cancer). For clarity, combination therapy does not require that two agents be administered together in a single composition or at the same time, although in some embodiments, two or more agents, including a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof) and a second agent described herein may be administered within the same period of time (e.g., within the same hour, day, week, or month).

The terms “cutoff” and “cutoff value” mean a value measured in an assay that defines the dividing line between two subsets of a population (e.g., likely responders and non-responders (e.g., responders and non-responders to a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof). In some instances, values that are equal to or above the cutoff value define one subset of the population, and values that are lower than the cutoff value define the other subset of the population. In other instances, values that are equal to or below the cutoff value define on subset of the population, and values above the cutoff value define the other. As described further below, the cutoff or cutoff value can define the threshold value.

As used herein, “diagnostic information” is information that is useful in determining whether a patient has a disease and/or in classifying (stratifying) the disease into a genotypic or phenotypic category or any category having significance with regard to the prognosis of the disease or its likely response to treatment (either treatment in general or any particular treatment described herein). In case of doubt, the present methods in which a biomarker or a plurality of biomarkers is assessed provide diagnostic information. Similarly, “diagnosis” refers to obtaining or providing any type of diagnostic information, including, but not limited to, whether a patient is likely to have or develop a disease; whether that disease has or is likely to reach a certain state or stage or to exhibit a particular characteristic (e.g., resistance to a therapeutic agent); information related to the nature or classification of a tumor; information related to prognosis (which may also concern resistance); and/or information useful in selecting an appropriate treatment (e.g., selecting THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof for a patient identified as having a cancer that is likely to respond to such an inhibitor or other treatment). A patient classified (i.e., stratified or selected) according to a method described herein and selected for treatment with a CDK7 inhibitor (including any of those just listed) is likely to respond well to the treatment, meaning that such a patient is more likely to be successfully treated than a patient with the same type of cancer who has not been so identified and is not in the same strata. Available treatments include therapeutic agents and other treatment modalities such as surgery, radiation, etc., and selecting an appropriate treatment encompasses the choice of withholding a particular therapeutic agent; the choice of a dosing regimen; and the choice of employing a combination therapy. Diagnostic information can be used to stratify patients and is thus useful in identifying and classifying a given patient according to, for example, biomarker status. Obtaining diagnostic information can constitute a step in any of the patient stratification methods described herein.

One of ordinary skill in the art will appreciate that the term “dosage form” may be used to refer to a physically discrete unit of an active agent (e.g., a therapeutic or diagnostic agent) for administration to a patient. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular patient is determined by one or more attending physicians and may involve administration of multiple dosage forms.

One of ordinary skill in the art will appreciate that the term “dosing regimen” may be used to refer to a set of unit doses (typically more than one) that are administered individually to a patient, separated by equal or unequal periods of time. A given therapeutic agent typically has a recommended dosing regimen, which may involve one or more doses, each of which may contain the same unit dose amount or differing amounts. In some embodiments, a dosing regimen comprises a first dose in a first dose amount, followed by one or more additional doses in a second dose amount that is different from the first dose amount. In some embodiments, a dosing regimen is correlated with a desired or beneficial outcome when administered across a relevant population (i.e., the regimen is a therapeutic dosing regimen).

As used herein, an “effective amount” of an agent (e.g., a chemical compound described herein, including any of the disclosed CDK7 inhibitors and pharmaceutically acceptable salts thereof), refers to an amount that produces or is expected to produce the desired effect for which it is administered. The effective amount will vary depending on factors such as the desired biological endpoint, the pharmacokinetics of the compound administered, the condition being treated, the mode of administration, and characteristics of the patient, as discussed further below and recognized in the art. The term can be applied to therapeutic and prophylactic methods. For example, a therapeutically effective amount is one that reduces the incidence and/or severity of one or more signs or symptoms of the disease. For example, in treating a cancer, an effective amount may reduce the tumor burden, stop tumor growth, inhibit metastasis or prolong patient survival. One of ordinary skill in the art will appreciate that the term does not in fact require successful treatment be achieved in any particular individual. Rather, a therapeutically effective amount is that amount that provides a particular desired pharmacological response in a significant number of patients when administered to patients in need of such treatment. In some embodiments, reference to a therapeutically effective amount may be a reference to an amount administered or an amount measured in one or more specific tissues (e.g., a tissue affected by the disease) or fluids (e.g., blood, saliva, serum, sweat, tears, urine, etc.). Effective amounts may be formulated and/or administered in a single dose or in a plurality of doses, for example, as part of a dosing regimen.

As used herein, an “enhancer” is a region of genomic DNA that helps regulate the expression of a gene and which can do so when positioned far away from the gene (e.g., up to about 1 Mbp away). An enhancer may overlap, but is often not composed of, gene coding regions. An enhancer is often bound by transcription factors and designated by specific histone marks. “Enhancer RNA” (eRNA) is an RNA that includes RNA transcribed from the DNA of an enhancer.

An identified patient can be “newly diagnosed” and therefore previously unexposed to a first agent (i.e.. a CDK7 inhibitor as described herein) or a second agent (i.e., a therapeutic agent described herein as useful in combination with a CDK7 inhibitor), in which case the patient may also be defined as treatment naive.

The term “patient” refers to any organism that is or may be subjected to a diagnostic method described herein or to which a compound described herein, or a pharmaceutically acceptable salt thereof, is or may be administered for, e.g., experimental, diagnostic, prophylactic, and/or therapeutic purposes. Typical patients include animals (e.g., mammals such as mice, rats, rabbits, non-human primates, and humans; domesticated animals, such as dogs and cats; and livestock or any other animal of agricultural or commercial value). A patient may be suffering from or susceptible to (i.e., have a higher than average risk of developing) a disease described herein and may display one or more signs or symptoms thereof.

The term “pharmaceutically acceptable,” when applied to a carrier used to formulate a composition disclosed herein (e.g., a pharmaceutical composition), means a carrier that is compatible with the other ingredients of the composition and not deleterious to a patient (e.g., it is non-toxic in the amount required and/or administered (e.g., in a unit dosage form)).

The term “pharmaceutically acceptable,” when applied to a salt form of a compound described herein, refers to a salt form that is, within the scope of sound medical judgment, suitable for use in contact with the tissues of humans (e.g., patients) and lower animals (including, but not limited to, mice and rats used in laboratory studies) without unacceptable toxicity, irritation, allergic response and the like, and that can be used in a manner commensurate with a reasonable benefit/risk ratio. Many pharmaceutically acceptable salts are well known in the art (see, e.g., Berge et al., J. Pharm. Sci. 66: 1-19, 1977). Pharmaceutically acceptable salts of the compounds (e.g., CDK7 inhibitors) described herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxyethanesulfonate, lactobionate, lactate, laurate, lauryl sulfate, MALAT1e, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, the term “population” means some number of items (e.g., at least 30, 40, 50, or more) sufficient to reasonably reflect the distribution, in a larger group, of the value being measured in the population. Within the context of the present invention, the population can be a discrete group of humans, laboratory animals, or cells lines (for example) that are identified by at least one common characteristic for the purposes of data collection and analysis. For example, a “population of samples” refers to a plurality of samples that is large enough to reasonably reflect the distribution of a value (e.g., a value related to the state of a biomarker) in a larger group of samples. The items in the population may be biological samples, as described herein. For example, each sample in a population of samples may be cells of a cell line or a biological sample obtained from a patient or a xenograft (e.g., a tumor grown in a mouse by implanting a tumorigenic cell line or a patient sample into the mouse). As noted, individuals within a population can be a discrete group identified by a common characteristic, which can be the same disease (e.g., the same type of cancer), whether the sample is obtained from living beings suffering from the same type of cancer or a cell line or xenograft representing that cancer.

The term “prevalence cutoff,” as used herein in reference to a specified value (e.g., the strength of a SE associated a biomarker disclosed herein) means the prevalence rank that defines the dividing line between two subsets of a population (e.g., a subset of “responders” and a subset of “non-responders,” which, as the names imply include patients who are likely or unlikely, respectively, to experience a beneficial response to a therapeutic agent or agents). Thus, a prevalence rank that is equal to or higher (e.g., a lower percentage value) than the prevalence cutoff defines one subset of the population; and a prevalence rank that is lower (e.g., a higher percentage value) than the prevalence cutoff defines the other subset of the population.

As used herein, the term “prevalence rank” for a specified value (e.g., the mRNA level of a specific biomarker) means the percentage of a population that are equal to or greater than that specific value. For example, a 35% prevalence rank for the amount of mRNA of a specific biomarker in a test cell means that 35% of the population have that level of biomarker mRNA or greater than the test cell.

The term “primary RNA transcript” as used herein refers to an RNA transcription product from a DNA sequence that includes a coding region of a gene (e.g., at least one exon) and/or a non-coding region of the gene (e.g., an intron or a regulatory region of the gene (e.g., an enhancer or super enhancer that regulates expression of the gene)). Thus, the primary RNA transcript can be an “enhancer RNA” or “eRNA,” a microRNA, a precursor mRNA (“pre-mRNA”) or mature mRNA. In methods of assessing the level of expression of a primary RNA transcript, one may assess a cDNA that has been synthesized or reverse transcribed from a primary RNA transcript.

As used herein, the terms “prognostic information” and “predictive information” are used to refer to any diagnostic information that may be used to indicate any aspect of the course of a disease or condition either in the absence or presence of treatment. Such information may include, but is not limited to, the average life expectancy of a patient, the likelihood that a patient will survive for a given amount of time (e.g., 6 months, 1 year, 5 years, etc.), the likelihood that a patient will be cured of a disease, the likelihood that a patient’s disease will respond to a particular therapy (wherein response may be defined in any of a variety of ways). Diagnostic information can be prognostic or predictive.

As used herein, the term “rank ordering” means the ordering of values from highest to lowest or from lowest to highest. In case of doubt, prevalence ranks can also be rank ordered.

As used herein, a “reference” refers to a standard (“reference standard”) or control relative to which a comparison is performed. For example, an agent, patient, population, sample, sequence, or value of interest is compared with a reference agent, patient, population, sample, sequence or value. The reference can be analyzed or determined substantially simultaneously with the analysis or determination of the item of interest or it may constitute a historical standard or control, determined at an earlier point in time and optionally embodied in a tangible medium. One of ordinary skill in the art is well trained in selecting appropriate references, which are typically determined or characterized under conditions that are comparable to those encountered by the item of interest. One of ordinary skill in the art will appreciate when sufficient similarities are present to justify reliance on and/or comparison to a particular possible reference as a standard or control.

As used herein, a “response” to treatment is any beneficial alteration in a patient’s condition that results from, or that correlates with, treatment. The alteration may be stabilization of the condition (e.g., inhibition of deterioration that would have taken place in the absence of the treatment), amelioration of, delay of onset of, and/or reduction in frequency of one or more signs or symptoms of the condition, improvement in the prospects for cure of the condition, greater survival time, and etc. A response may be a patient’s response or a tumor’s response.

As used herein, when the term “strength” is used to refer to a portion of an enhancer or a SE, it means the area under the curve of the number of H3K27Ac or other genomic marker reads plotted against the length of the genomic DNA segment analyzed. Thus, “strength” is an integration of the signal resulting from measuring the mark at a given base pair over the span of the base pairs defining the region being chosen to measure.

As used herein, the term “super-enhancer” (SE) refers to a subset of enhancers that contain a disproportionate share of histone marks and/or transcriptional proteins relative to other enhancers in a particular cell or cell type. Genes regulated by SEs are predicted to be of high importance to the function of a cell. SEs are typically determined by rank ordering all of the enhancers in a cell based on strength and determining, using available software such as ROSE (bitbucket.org/young__ computation/rose), the subset of enhancers that have significantly higher strength than the median enhancer in the cell. As needed, one of ordinary skill in the art can consult, e.g., U.S. Pat. No. 9,181,580, which describes methods of identifying SEs that modulate the expression of cell type-specific genes (e.g., genes that define the identity of embryonic stem cells) and which is hereby incorporated by reference herein in its entirety.

The terms “threshold” and “threshold level” mean a level that defines the dividing line between two subsets of a population (e.g., responders and non-responders). A threshold or threshold level can define a prevalence cutoff or a cutoff value and may be assessed with regard to various features of a biomarker (e.g., the level, ordinal rank, or prevalence rank of primary RNA transcripts expressed from the biomarker gene or the strength, ordinal rank, or prevalence rank of a super enhancer associated with the biomarker gene).

As used herein, the terms “treatment,” “treat,” and “treating” refer to reversing, alleviating, delaying the onset of, and/or inhibiting the progress of a “pathological condition” (e.g., a disease, such as cancer) described herein. In some embodiments, “treatment,” “treat,” and “treating” require that signs or symptoms of the disease have developed or have been observed. In other embodiments, treatment may be administered in the absence of signs or symptoms of the disease or condition (e.g., in light of a history of symptoms and/or in light of genetic or other susceptibility factors). Treatment may also be continued after symptoms have resolved, for example, to delay or inhibit recurrence.

As the invention relates to compositions and methods for diagnosing and treating patients who have cancer, the terms “active agent,” “anti-cancer agent,” “pharmaceutical agent,” and “therapeutic agent” are used interchangeably (unless the context clearly indicates otherwise) and CDK7 inhibitors (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof) would be understood by one of ordinary skill in the art as active, anti-cancer, pharmaceutical, or therapeutic agents. As noted, the treatment methods and uses encompass combination therapies/uses in which a CDK7 inhibitor, including any of those just listed, is administered or used in combination with one or more additional agents (e.g., an additional anti-cancer therapeutic), as described herein. In keeping with convention, in any embodiment requiring two agents, we may refer to one as the “first” agent and to the other as the “second” agent to underscore that the first and second agents are distinct from one another. The designation “first” need not be explicit It is to be understood that where two entities (e.g., two therapeutic or anti-cancer agents) are described, one may constitute the “first” agent and the other may constitute the “second” agent.

The invention also features kits that include a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof) and instructional materials that describe a suitable/identified patient, methods of identifying such a patient for treatment (e.g., by any one of the diagnostic stratification methods described herein), and/or instructions for administering the CDK7 inhibitor alone or in combination with at least one other therapeutic agent (e.g., an additional/second anti-cancer therapeutic). By “kit” we mean a set of articles needed for a specific purpose, as conventionally known in the art. The kits of the invention can also include a second agent (e.g., an anti-cancer agent), including any one or more of the second agents described herein and instructions for use in a population of patients identified as described. In keeping with convention, the kits of the invention comprise a set of articles needed for a specific purpose. Each article (e.g., a first agent) can be contained within a container, and a plurality of containers can be physically united within a package.

As indicated, each therapeutic method and any diagnostic method that employs a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof) or a composition (e.g., a pharmaceutical composition containing any one or more of the CDK7 inhibitors just listed) may also be expressed in terms of use and vice versa. For example, the invention encompasses the use of a compound or composition described herein for the treatment of a disease described herein (e.g., cancer); a compound or composition for use in diagnosing and/or treating or a disease (e.g., cancer); and the use of the compound or composition for the preparation of a medicament for treating a disease described herein (e.g., cancer).

The methods of the invention that concern diagnosing and/or treating a cancer described herein (or “use” of a covalent or non-covalent CDK7 inhibitor for such purpose) may specifically exclude any one or more of the types of cancers described herein. For example, the invention features methods of treating cancer by administering a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof), with the proviso that the cancer is not a colorectal cancer; with the proviso that the cancer is not a CRC or lung cancer; with the proviso that the cancer is not a CRC, lung cancer, and/or pancreatic cancer; and so forth, with exclusions selected from any of the cancer types disclosed herein and with the same notion of variable exclusion from lists of elements relevant to other aspects of the invention (e.g., chemical substituents of compounds or components of kits and pharmaceutical compositions).

In one aspect, the invention features methods of diagnosing a patient by determining, in a biological sample obtained from the patient, whether (a) a RAS gene (e.g., KRAS) is mutated or genetically amplified (e.g., by virtue of a copy number increase), contains an epigenetic alteration (i.e., a functionally relevant change to the genome that does not involve a change in nucleotide sequence (e.g., DNA methylation or histone modulation)), is translocated, is transcribed at a level equal to or above a pre-determined threshold (possibly due to association with a super-enhancer), or encodes a protein that is mutant, translated at a level equal to or above a pre-determined threshold, or has increased activity relative to a reference standard; and/or (b) chromosomal band 9q34 is completely or partially deleted. A partial deletion can be detected by, for example, loss of at least two resident genes (e.g., at least 2, 5, 10, 20, or 30 or more resident genes). In either event (a or b), the patient is thereby diagnosed and identified as a good candidate for treatment with a CDK7 inhibitor, including any one or more of those described herein. In embodiments of these methods, the patient can be a human. The human genome carries three RAS genes: HRAS (encoding HRAS), NRAS (encoding NRAS), and KRAS (encoding KRAS4a and KRAS4b, resulting from alternative splicing; see Barbacid, Ann. Rev. Biochem. 56:779-827, 1987)). When KRAS is the biomarker, the biological sample can be analyzed for the presence of a KRAS gain-offunction mutation, resulting in overactive/prolonged binding between KRAS and GTP that drives downstream effectors that contribute to cell cycle dysregulation.

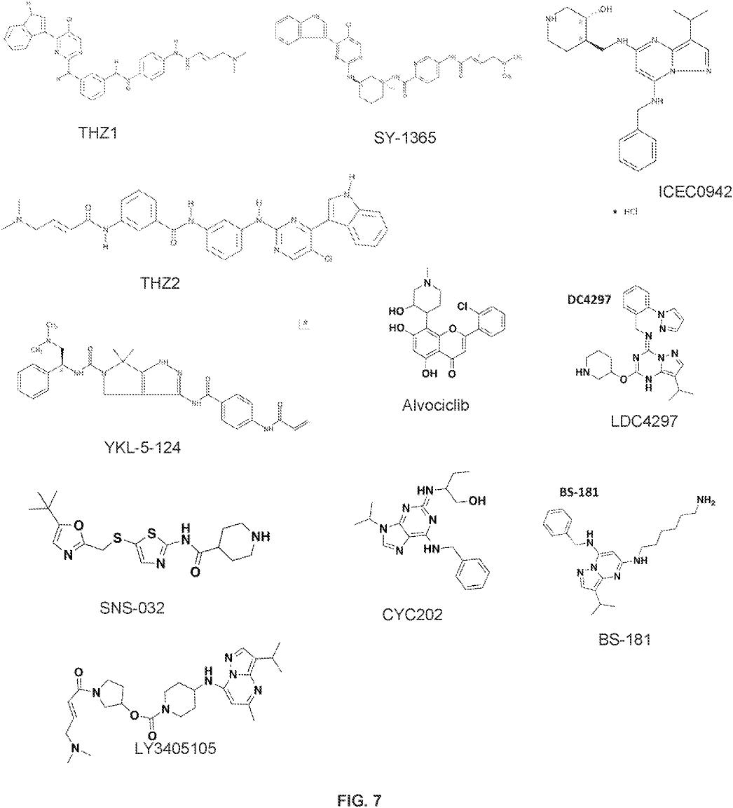

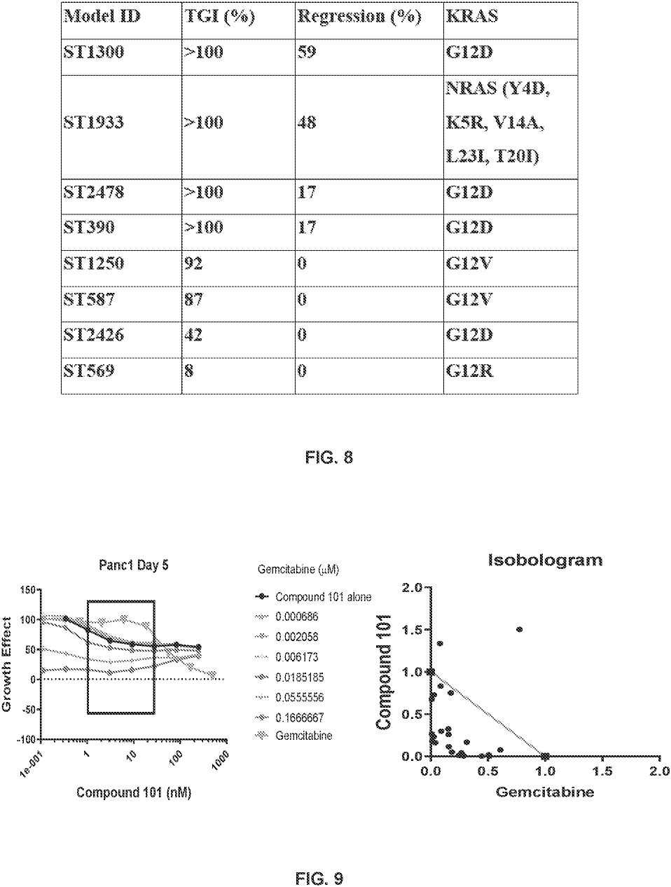

In another aspect, the invention features methods of diagnosing and treating a patient as described herein and, as noted, methods of treatment may be expressed in terms of a “use” for a compound or pharmaceutical composition described herein. For example, the invention features the use of a CDK7 inhibitor as known in the art and/or shown, for example, in FIG. 7, or a compound of Formula (I), (Ia), a species thereof, or a pharmaceutically acceptable salt thereof in treating cancer in a patient who has been identified by a diagnostic method described herein. For example, the present treatment methods include administering a CDK7 inhibitor to a patient who has been identified by virtue of having: (a) a RAS gene (e.g., KRAS) that is mutated (with the mutations including, but not being limited to, those shown in FIG. 8) or genetically amplified (e.g., by virtue of a copy number increase), contains an epigenetic alteration (i.e., a functionally relevant change to the genome that does not involve a change in nucleotide sequence (e.g., DNA methylation or histone modulation)), is translocated, is transcribed at a level equal to or above a pre-determined threshold (possibly due to association with a super-enhancer), or encodes a protein that is mutant, translated at a level equal to or above a pre-determined threshold, or has increased activity relative to a reference standard; and/or (b) a complete or partial deletion of chromosomal band 9q34.

In any of the methods of administering a CDK7 inhibitor or uses thereof, as described herein, the CDK7 inhibitor can be THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof. For example, the CDK7 inhibitor can be a compound of Formula (I):

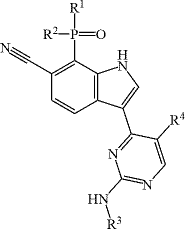

or a pharmaceutically acceptable salt thereof, optionally within a pharmaceutical composition, wherein:
R¹ is methyl or ethyl;
R² is methyl or ethyl;
R³ is 5-methylpiperidin-3-yl, 5,5-dimethylpiperidin-3-yl, 6-methylpiperdin-3-yl, or 6,6-dimethylpiperidin-3-yl, wherein one or more hydrogen atoms in R³ is optionally replaced by deuterium; and
R⁴ is —CF₃ or chloro. Where the compound conforms to Formula (I), (i) R¹ can be methyl and R² can be methyl or (ii) R¹ can be methyl and R² can be ethyl. In any of these embodiments, R⁴ can be —CF₃ or chloro; preferably, R⁴ is —CF₃. In any of these embodiments, R³ can be 5-methylpiperidin-3-yl, wherein one or more hydrogen atoms in R³ is optionally replaced by deuterium; 5,5-dimethylpiperidin-3-yl, wherein one or more hydrogen atoms in R³ is optionally replaced by deuterium; 6-methylpiperdin-3-yl, wherein one or more hydrogen atoms in R³ is optionally replaced by deuterium; or 6,6-dimethylpiperidin-3-yl, wherein one or more hydrogen atoms in R³ is optionally replaced by deuterium. The CDK7 inhibitor can have structural Formula (Ia):

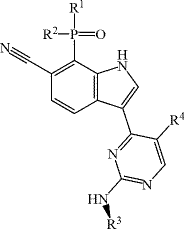

or be the pharmaceutically acceptable salt thereof, wherein R³ is

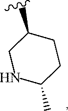

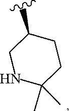

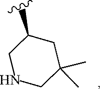

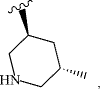

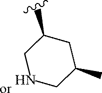

and R¹, R², and R⁴ are as described herein. As noted, in certain embodiments, the proliferative disease to be treated or prevented using a composition of the invention is cancer. All types of cancers disclosed herein or known in the art are contemplated as being within the scope of the invention, but particularly those that are known to be associated with CDK7 activity (e.g., CDK7 overactivity, overexpression, or misexpression). Thus, in addition to the biomarker status, methods of the invention can also be carried out when a biological sample from a patient has been determined to include cancer cells associated with CDK7 activity. In embodiments, the patient has been determined to have a cancer in which a KRAS gene is mutated, is genetically amplified, contains an epigenetic alteration, is translocated, is transcribed at a level equal to or above a pre-determined threshold, or encodes a protein that is mutant, translated at a level equal to or above a pre-determined threshold, or has increased activity relative to a reference standard; and in which one or more of the following, additional biomarkers have been determined to be positive: BCL2L1, BRAF, DIS3, WNT; 1p36, msi, 8q, TP53, and 20q.

In certain embodiments, the proliferative disease is a blood cancer, which may also be referred to as a hematopoietic or hematological cancer or malignancy. The blood cancer, including any of the specific types listed below, can be determined to be “positive” for a biomarker described herein and, optionally, associated with CDK7 overexpression, misexpression, or overactivity (e.g., relative to a reference standard). More specifically and in various embodiments, the blood cancer can be a leukemia such as acute lymphocytic leukemia (ALL; e.g., B cell ALL or T cell ALL), acute myelocytic leukemia (AML; e.g., B cell AML or T cell AML), chronic myelocytic leukemia (CML; e.g., B cell CML or T cell CML), chronic lymphocytic leukemia (CLL; e.g., B cell CLL (e.g., hairy cell leukemia) or T cell CLL), chronic neutrophilic leukemia (CNL), or chronic myelomonocytic leukemia (CMML). Where the cancer is AML, it may be undifferentiated acute myelblastic leukemia (M0), acute myeloblastic leukemia with minimal maturation (M1), acute myeloblastic leukemia with maturation (M2), acute promyelocytic leukemia (APL/M3), acute myelomonocytic leukemia (M4), or acute myelomonocytic leukemia with eosinophilia (M5). The blood cancer can also be a lymphoma such as Hodgkin lymphoma (HL, e.g., B cell HL. or T cell HL), non-Hodgkin lymphoma (NHL, which can be deemed aggressive; e.g., B cell NHL or T cell NHL), follicular lymphoma (FL), chronic lymphocytic leukemia/small lymphocytic lymphoma (CLL/SLL), mantle cell lymphoma (MCL), a marginal zone lymphoma (MZL), such as a B cell lymphoma (e.g., splenic marginal zone B cell lymphoma), primary mediastinal B cell lymphoma (e.g., splenic marginal zone B cell lymphoma), primary mediastinal B cell lymphoma, Burkitt lymphoma (BL), lymphoplasmacytic lymphoma (i.e., Waldenstrom’s macroglobulinemia), immunoblastic large cell lymphoma, precursor B lymphoblastic lymphoma, or primary central nervous system (CNS) lymphoma. The B cell NHL can be diffuse large cell lymphoma (DLCL; e.g., diffuse large B cell lymphoma (DLBCL; e.g., germinal center B cell-like (GCB) DLBCL or activated B-cell like (ABC) DLBCL)), and the T cell NHL can be precursor T lymphoblastic lymphoma or a peripheral T cell lymphoma (PTCL). In turn, the PTCL can be a cutaneous T cell lymphoma (CTCL) such as mycosis fungoides or Sezary syndrome, angioimmunoblastic T cell lymphoma, extranodal natural killer T cell lymphoma, enteropathy type T cell lymphoma, subcutaneous anniculitis-like T cell lymphoma, or anaplastic large cell lymphoma. While the invention is not limited to treating or preventing blood cancers having any particular cause or presentation, stem cells within the bone marrow may proliferate, thereby becoming a dominant cell type within the bone marrow and a target for a compound described herein. Leukemic cells can accumulate in the blood and infiltrate organs such as the lymph nodes, spleen, liver, and kidney. In some embodiments, a compound of the present disclosure or a specified form thereof is useful in the treatment or prevention of a leukemia or lymphoma.

In other embodiments, the proliferative disease is characterized by a solid tumor considered to be either of its primary location or metastatic. Cancer cells within the solid tumor, including any of the specific tumor types listed below, can be determined to be “positive” for a biomarker described herein and, optionally, associated with CDK7 overexpression, misexpression, or overactivity (e.g., relative to a reference standard). For example, in various embodiments, the cancer or tumor that is amenable to treatment and which is treated or prevented as described herein is an acoustic neuroma; adenocarcinoma; adrenal gland cancer; anal cancer; angiosarcoma (e.g., lymphangiosarcoma, lymphangio-endotheliosarcoma, hemangiosarcoma); appendix cancer; benign monoclonal gammopathy (also known as monoclonal gammopathy of unknown significance (MGUS); biliary cancer (e.g., cholangiocarcinoma); bladder cancer; breast cancer (e.g., adenocarcinoma of the breast, papillary carcinoma of the breast, mammary cancer, medullary carcinoma of the breast, or breast invasive carcinoma; any of which may be present in subjects having a particular profile, such as an HR+ (ER+ or PR+), HER2+, HR- (having neither estrogen nor progesterone receptors), a triple negative breast cancer (TNBC; ER-/PR-/HER2-), or a triple-positive breast cancer (ER+/PR+/HER2+); a brain cancer (e.g., meningioma, glioblastoma, glioma (e.g., astrocytoma, oligodendroglioma), medulloblastoma); bronchus cancer; carcinoid tumor, which may be benign; cervical cancer (e.g., cervical adenocarcinoma); choriocarcinoma, chordoma; craniopharyngioma; a cancer present in the large intestine, such as colorectal cancer (CRC, e.g., colon cancer, rectal cancer, or colorectal adenocarcinoma); connective tissue cancer; epithelial carcinoma; ependymoma; endothelio-sarcoma (e.g., Kaposi’s sarcoma or multiple idiopathic hemorrhagic sarcoma); endometrial cancer (e.g., uterine cancer, uterine sarcoma); esophageal cancer (e.g., adenocarcinoma of the esophagus, Barrett’s adenocarcinoma); Ewing’s sarcoma (or other pediatric sarcoma, such as embryonal rhabdomyosarcoma or alveolar rhabdomyosarcoma); eye cancer (e.g., intraocular melanoma, retinoblastoma); familiar hypereosinophilia; gallbladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hypopharynx cancer; inflammatory myofibroblastic tumors; immunocytic amyloidosis; kidney cancer (e.g., nephroblastoma a.k.a. Wilms’ tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma, squamous cell carcinoma, large cell carcinoma of the lung, or lung squamous cell carcinoma); leiomyosarcoma (LMS); mastocytosis (e.g., systemic mastocytosis); mouth cancer; muscle cancer; myelodys-plastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer), ovarian cancer (e.g., cystadenocarcinoma, ovarian embryonal carcinoma, ovarian adenocarcinoma, HGSOC, LGSOC, epithelial ovarian cancer (e.g., ovarian clear cell carcinoma or mucinous carcinoa), sex cord stromal tumors (granulosa cell), endometroid tumors, or ovarian serous cystadenocarcinoma); papillary adenocarcinoma; pancreatic cancer (whether an exocrine tumor (e.g., pancreatic adenocarcinoma, PDAC), intraductal papillary mucinous neoplasm (IPMN), or a neuroendocrine tumor (e.g., PNETs or islet cell tumors); penile cancer (e.g., Paget’s disease of the penis and scrotum); pinealoma; primary peritoneal cancer, primitive neuroectodermal tumor (PNT); plasma cell neoplasia; paraneoplastic syndromes; prostate cancer, which may be castration-resistant (e.g., prostate adenocarcinoma); rhabdomyosarcoma; salivary gland cancer; skin cancer (e.g., squamous cell carcinoma (SCC), keratoacanthoma (KA), melanoma, basal cell carcinoma (BCC)); small bowel or small intestine cancer; soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget’s disease of the vulva). We use the term “gastrointestinal (GI) tract cancer” to refer to a cancer present anywhere in the GI tract, including cancers of the mouth, throat, esophagus, stomach, large or small intestine, rectum, and anus. In some embodiments, the proliferative disease is associated with pathologic angiogenesis, and the methods of the invention and uses of a compound described herein (or any specified form thereof) encompass inhibiting pathologic angiogenesis in the context of cancer treatment (e.g., of a blood cancer or solid tumor). As noted above, the cancer can be a neuroendocrine cancer, and such tumors can be treated as described herein regardless of the organ in which they present.

Such a patient can be: treated with a platinum-based therapeutic agent (e.g., carboplatin or oxaliplatin) as a second agent; a patient whose cancer has developed resistance to a platinum-based therapeutic agent (e.g., carboplatin or oxaliplatin); or a patient undergoing treatment with a CDK4/6 inhibitor used alone or in combination with one or more of an aromatase inhibitor, a selective estrogen receptor modulator or a selective estrogen receptor degrader. The patient’s cancer may have become resistant to the CDK4/6 inhibitor or at risk of becoming so. In the context of the uses described here (e.g., where the patient has been selected by virtue of having a level of BLC2-like mRNA equal to or below the pre-determined threshold level), the cancer can be a breast cancer (e.g., a triple negative breast cancer (TNBC), an ovarian cancer, a lung cancer (e.g., non-small cell lung cancer), or a blood cancer (e.g., acute myeloid leukemia (AML) or a subtype thereof).

The patient can be one who has undergone, is presently undergoing, or who will undergo (e.g., has been prescribed) treatment with a Bcl-2 inhibitor, such as venetoclax.

In another aspect, the invention features the use of a CDK7 inhibitor, including a compound of Formula (I), (Ia), a species thereof, or a pharmaceutically acceptable salt thereof, in treating a patient identified as described herein, with a combination therapy with an effective amount of a second agent in treating a patient who has cancer, wherein: (a) the cancer is TNBC, an estrogen receptor-positive (ER⁺) breast cancer, pancreatic cancer, or a squamous cell cancer of the head or neck; and the second agent is a CDK4/6 inhibitor; (b) the cancer is a breast cancer, or an ovarian cancer; and the second agent is a PARP inhibitor; (c) the cancer is AML; and the second agent is a FLT3 inhibitor; (d) the cancer is an ovarian cancer; and the second agent is a platinum-based anti-cancer agent; (e) the cancer is TNBC, AML, Ewing’s sarcoma, or an osteosarcoma; and the second agent is a BET inhibitor; (f) the cancer is TNBC, AML, an ovarian cancer, or non-small cell lung cancer; and the second agent is a Bcl-2 inhibitor. In particular embodiments, the cancer is AML and the second agent is a Bcl-2 inhibitor, such as venetoclax; the cancer is an epithelial ovarian cancer, a fallopian tube cancer, a primary peritoneal cancer, a triple negative breast cancer or a Her2⁺/ER⁻/PR⁻ breast cancer and the second agent is a PARP inhibitor, such as olaparib or niraparib; the cancer is an ovarian cancer and the second agent is a platinum-based anti-cancer agent, such as carboplatin or oxaliplatin.

With regard to combination therapies, a patient identified as described herein can be treated with a combination of a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof) and second agent that can be, but is not limited to, a Bcl-2 inhibitor such as APG-1252, APG-2575, BP1002 (prexigebersen), the antisense oligonucleotide known as oblimersen (G3139), S55746/BCL201, or venetoclax (e.g., venetoclax tablets marketed as Venclexta®); a CDK9 inhibitor such as alvocidib/DSP-2033/flavopiridol, AT7519, AZD5576, BAY1251152, BAY1 143572, CYC065, nanoflavopiridol, NVP2, seliciclib (CYC202), TG02, TP-1287, VS2-370 or voruciclib (formerly P1446A-05); a hormone receptor (e.g., estrogen receptor) degradation agent, such as fulvestrant (e.g., marketed as Faslodex® and others); a Flt3 (EMS-like tyrosine kinase 3) inhibitor such as CDX-301, CG′806, CT053PTSA, crenolanib (e.g., crenolanib besylate), ENMD-2076, FF-10101-01, FLYSYN, gilteritinib (ASP2215), HM43239, lestautinib, ponatinib (e.g., marketed as Iclusig®, previously AP24534), NMS-088, sorafenib (e.g., marketed as Nexavar®), sunitinib, pacritinib, pexidartinib/PLX3397, quizartinib, midostaurin (e.g., marketed as Rydapt®), SEL24, SKI-G-801, or SKLB1028; a PARP inhibitor such as olaparib (e.g., marketed as Lynparza®), rucaparib (e.g., marketed as Rubraca®), talaroparib (e.g., marketed as Talzenna®), veliparib (ABT-888), or niraparib (e.g., marketed as Zejula®); a BET inhibitor such as ABBV-075, BAY-299, BAY-1238097, BMS-986158, CPI-0610, CPI-203, FT-1101, GS-5829, GSK-2820151, GSK-525762, I-BET151, I-BET762, INCB054329, JQ1, MS436, OTX015 (see U.S. Pat. No. 8,476,260, which is hereby incorporated by reference herein in its entirety), PFI-1, PLX51107, RVX2135, TEN-010, ZEN-3694; a platinum-based therapeutic agent such as cisplatin, oxaliplatin (e.g., marketed as Eloxatin®), nedaplatin, carboplatin (e.g., marketed as Paraplatin®), phenanthriplatin, picoplatin, satraplatin (JM216), or triplatin tetranitrate, a CDK4/6 inhibitor such as BPI-1 178, G1T38, palbociclib (e.g., marketed as Ibrance®), ribociclib (e.g., marketed as Kisqali®), ON 123300, trilaciclib, or abemaciclib (e.g., marketed as Verzenio®); a MEK inhibitor such as trametinib (e.g., marketed as Mekinist®), binimetinib, or selumetinib; or a phosphoinositide 3-kinase (P13 kinase) inhibitor, optionally of Class I (e.g., Class IA) and/or optionally directed against a specific PI3K isoform. The PI3K inhibitor can be idelalisib (e.g., marketed as Zydelig®), copanlisib (e.g., marketed as Aliqopa®), duvelisib (e.g., marketed as Copiktra®), or alpelisib (e.g., marketed as Piqray®). In other embodiments, the additional/second agent can be capecitabine (e.g., marketed as Xeloda®). In other embodiments, the additional/second agent can be a KRAS inhibitor such as MRTX849 (Mirati Therapeutics, Inc.), AMG510 (Amgen) or BI 1701963 (Boerhinger Ingelheim). In other embodiments, the additional/second agent can be an ERK inhibitor such as LY3214996. In other embodiments, the additional/second agent can be a BRAF inhibitor such as encorafenib (Braftovi®), dabrafenib (Tafinlar®), and vemurafenib (Zelboraf®). Where a CDK7 inhibitor is administered with a BRAF inhibitor, the patient may be suffering from a skin cancer (e.g., melanoma) or endocrine cancer (e.g., thyroid cancer).

APG-1252 is a dual Bcl-2Bcl-xL inhibitor that has shown promise in early clinical trials when patients having SCLC or another solid tumor were dosed between 10-400 mg (e.g., 160 mg) intravenously twice weekly for three weeks in a 28-day cycle (see Lakhani et al., J. Clin. Oncol. 36:15_suppl, 2594, and ClinicalTrials.gov identifier NCT03080311). APG-2575 is a Bcl-2 selective inhibitor that has shown promise in preclinical studies of FL and DLBCL in combination with ibrutinib (see Fang et al., AACR Annual Meeting 2019, Cancer Res. 79(13 Suppl):Abstract No. 2058) and has begun clinical trials as a single-agent treatment for patients with blood cancers; in a dose escalation study, patients are given 20 mg, once daily, by mouth, for four consecutive weeks as one cycle. Escalations to 50, 100, 200, 400, 600 and 800 mg are planned to identify the MTD (see ClinicalTrials.gov identifier NCT03537482). BP1002 is an uncharged P-ethoxy antisense oligodeoxynucleotide targeted against Bcl-2 mRNA that may have fewer adverse effects than other antisense analogs and has shown promise in inhibiting the growth of human lymphoma cell lines inclubated with BP1002 for four days and of CJ cells (transformed FL cells) implanted into SCID mice (see Ashizawa et al., AACR Annual Meeting 2017, Cancer Res. 77(13 Suppl):Abstract No . 5091). BP1002 has also been administered in combination with cytarabine (LDAC) to patients having AML (see ClinicalTrials.gov identifier NCT04072458). 555746/FiCL,201 is an orally available, selective Bcl-2 inhibitor that, in mice, demonstrated anti-tumor efficacy in two blood cancer xenograft models (Casara et al., Oncotarget 9(28):20075-88, 2018). A phase I dose-escalation study was designed to administer film-coated tablets containing 50 or 100 mg of S55746, in doses up to 1500 mg, to patients with CLL or a B cell NHL including FL, MCL, DLBCL, SLL, MZL, and MM (see ClinicalTrials.gov identifier NCT02920697). Venetoclax tablets have been approved for treating adult patients with CLL or SLL and, in combination with azacytidine, or decitabine, or low-dose cytarabine, for treating newly-diagnosed AML, in patients who are at least 75 years old or who have comorbidities that preclude the use of intensive induction chemotherapy. Dosing for CLL/SLL can follow the five-week ramp-up schedule and dosing for AML can follow the four-day ramp-up, both described in the product insert, together with other pertinent information. Should one of ordinary skill in the art require additional guidance, resources include U.S. Pat. No. 8,546,399, which describes, inter alia, methods of making venetoclax and formulations containing it; U.S. Pat. No. 9,174,982, which describes, inter alia, methods of using venetoclax; and U.S. Pat. No. 9,539,251, which describes, inter alia, methods of using venetoclax in combination with a second therapeutic agent to treat cancer. Each of these patents is hereby incorporated by reference in its entirety. Alvocidib was studied in combination with cytarabine/mitoxantrone or cytarabine/daunorubicin in patients with AML, with the details of administration being available at ClinicalTrials.gov with the identifier NCT03563560 (see also Yeh et al., Oncotarget 6(5):2667-2679, 2015, Morales et al., Cell Cycle 15(4):519-527, 2016, and Zeidner et al., Haematologica 100(9):1172-1179, 2015). AT7519 has been administered in a dose escalation format to eligible patients having refractory solid tumors. While there was some evidence of clinical activity, the appearance of QTc prolongation precluded further development at the dose schedule described by Mahadevan et al. (J. Clin. Oncol. ASCO Abstract No. 3533; see also Santo et al., Oncogene 29:2325-2336, 2010, describing the preclinical activity of AT7519 in MM). AZD5576 induced apoptosis in breast and lung cancer cell lines at the nanomolar level (see Li et al., Bioorg. Med. Chem. Lett. 27(15):3231-3237, 2017) and has been examined alone and in combination with acalabrutinib for the treatment of NHL (see AACR 2017 Abstract No. 4295). BAY1 251152 was the subject of a phase I clinical trial to characterize the MTD in patients with advanced blood cancers; the agent was infused weekly in 21-day cycles (see ClinicalTrials.gov identifier NCT02745743; see also Luecking et al., AACR 2017 Abstract No. 984). Voruciclib is a clinical stage oral CDK9 inhibitor that represses MCL-1 and sensitizes high-risk DLBCL to BCL2 inhibition. Dey et al. (Scientific Reports 7:18007, 2017) suggest that the combination of voruciclib and venetoclax is promising for a subset of high-risk DLBCL patients (see also ClinicalTrials.gov identifier NCT03547115). Fulvestrant has been approved for administration to postmenopausal women with advanced hormone receptor (HR)-positive, HER2-negative breast cancer, with HR-positive metastatic breast cancer whose disease progressed after treatment with other anti-estrogen therapies, and in combination with palbociclib (Ibrance®). Fulvestrant is administered by intramuscular injection at 500 or 250 mg (the lower dose being recommended for patients with moderate hepatic impairment) on days 1, 15, and 29, and once monthly thereafter (see the product insert for additional information . One can also consult, as needed, U.S. Pat. Nos. 6,774,122, 7,456,160, 8,329,680, and 8,329,680, which describe, inter alia, formulations comprising fulvestrant for, e.g., sustained release and intramuscular injection, Each of these patents is hereby incorporated by reference herein in its entirety. Ponatinib has been administered in clinical trials to patients with CML or ALL (see ClinicalTrials.gov identifiers NCT0066092072, NCT012074401973, NCT02467270, NCT03709017, NCT02448095, NCT03678454, and NCT02398825) as well as solid tumors, such as biliary cancer and NSCLC (NCT02265341, NCT02272998, NCT01813734, NCT02265341, NCT02272998, NCT01813734, NCT02265341, NCT02272998, NCT01813734, NCT01935336, NCT03171389, and NCT03704688; see also the review article by Tan et al., Onco. Targets Ther. 12:635-645, 2019). Additional information regarding the dosing regimen can be found in the product insert; see also U.S. Pat. Nos. 8,114,874; 9,029,533, and 9,493,470, which describe synthesis methods, formulations, and indications for ponatinib and each of which is hereby incorporated by reference herein in its entirety. Sorafenib has been approved for the treatment of kidney and liver cancers, AML, and radioactive iodine resistant advanced thyroid cancer, and a clinical trial was initiated in patients with desmoid-type fibromatosis (see ClinicalTrials.gov identifier NCT02066181). Information regarding dosage can be found in the product insert, which advises administration of two, 400 mg tablets twice daily; see also U.S. Pat. Nos. 7,235,576; 7,351,834, 7,897,623; 8,124,630; 8,618,141; 8,841,330; 8,877,933; and 9,737,488, each of which is hereby incorporated by reference herein in its entirety. Midostaurin has been administered to patients having AML, MDS, or systemic mastocytosis, and has been found to significantly prolong survival of FLT3-mutated AML patients when combined with conventional induction and consolidation therapies (see Stone et al., ASH 57th Annual Meeting, 2015 and Gallogly et al., Ther. Adv. Hematol. 8(9):245-251, 2017; clin see also the product insert, ClinicalTrials.gov identifier NCT03512197, and U.S. Pat. Nos. 7,973,031; 8,222,244; and 8,575,146, each of which is hereby incorporated by reference herein in its entirety. The information provided here and publicly available can be used to practice the methods and uses of the invention. In case of doubt, the invention encompasses combination therapies that require a compound of the invention or a pharmaceutically acceptable salt thereof and any one or more additional/second agents, which may be administered at or below a dosage currently approved for single use (e.g., as described above), to a patient as described herein.

Where the combination therapy employs a compound of the invention and: a CDK4/6 inhibitor, the patient can have a breast cancer (e.g., TNBC or an ER+ breast cancer), pancreatic cancer, lung cancer (e.g., SCLC or NSCLC), or squamous cell cancer of the head and neck; a CDK9 inhibitor, the patient can have a breast cancer and, more specifically, a Her2⁺/ER⁻/PR⁻ breast cancer; a Flt3 inhibitor (e.g., midostaurin), the patient can have a hematological cancer (e.g., AML); a BET inhibitor, the patient can have a hematological cancer (e.g., AML), a breast cancer (e.g., TNBC), an osteosarcoma or Ewing’s Sarcoma; a Bcl-2 inhibitor (e.g., venetoclax), the patient can have a breast cancer (e.g., TNBC), an ovarian cancer, a lung cancer (e.g., NSCLC) or a hematological cancer (e.g., AML); or a PARP inhibitor (e.g., niraparib or olaparib), the patient can have a breast cancer (e.g., TNBC or Her2⁺/ER⁻/PR⁻ breast cancer), an ovarian cancer (e.g., an epithelial ovarian cancer), a fallopian tube cancer, or a primary peritoneal cancer.

The invention provides pharmaceutical kits for treating cancer comprising a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof) and, optionally, a second therapeutic agent selected from: (a) a Bcl-2 inhibitor, (b) a CDK9 inhibitor, (c) a Flt3 inhibitor, (d) a PARP inhibitor, (e) a BET inhibitor, or (f) a CDK4/6 inhibitor, any of which may be selected from those disclosed herein. The kit can include optional instructions for: (a) reconstituting (if necessary) the CDK7 inhibitor (as just listed) and/or the second therapeutic agent; (b) administering each of the CDK7 inhibitor and/or the second therapeutic agent; and/or (c) a list of specific cancers for which the kit is useful or diagnostic methods by which they may be determined (these methods including those described herein for patient selection based on the status of a biomarker described herein). The kit can also include any type of paraphernalia useful in administering the active agent(s) contained therein (e.g., tubing, syringes, needles, sterile dressings, tape, and the like).

The invention provides a method of treating a cancer in a human patient by administering to the patient a combination of a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof) and a platinum-based standard of care (SOC) anti-cancer agent for such cancer or a taxane In one embodiment, the cancer is of a reproductive organ (e.g., an ovarian cancer); the SOC anti-cancer agent is a platinum-based anti-cancer agent (e.g., carboplatin, cisplatin, or oxaliplatin); and the CDK7 inhibitor is a compound of Formula (I), (Ia), a species thereof, or a pharmaceutically acceptable salt thereof. In some embodiments, the human patient is, has been determined to be, or has become resistant (after some initial responsiveness) to the platinum-based anti-cancer agent when administered as either a monotherapy or in combination with an anti-cancer agent other than a CDK7 inhibitor. In some aspects of this embodiment, the human patient is determined to have become resistant to the platinum-based anti-cancer agent when administered as a monotherapy or in combination with an anti-cancer agent other than a CDK7 inhibitor after some initial efficacy of that prior treatment. In some aspects of this embodiment, the SOC anti-cancer agent is a taxane (e.g., paclitaxel).

The invention provides a method of treating HR⁺ breast cancer in a human patient selected on the basis of being resistant to treatment with a CDK4/6 inhibitor comprising the step of administering to the patient a compound of Formula (I), (Ia), a species thereof, or a pharmaceutically acceptable salt thereof. In some embodiments, prior to administration of the compound of Formula (I), (la), a species thereof, or a pharmaceutically acceptable salt thereof, the patient is, has been determined to be, or has become resistant (after some initial responsiveness) to a prior treatment with a CDK4/6 inhibitor alone or in combination with another SOC agent for breast cancer other than a CDK7 inhibitor, such as an aromatase inhibitor (e.g., letrozole, anastrozole) or a SERM or SERD such as tamoxifen or fulvestrant. In other words, the identified patient is selected for treatment with a compound of Formula (I), (Ia), a species thereof, or a pharmaceutically acceptable salt thereof on the basis of being resistant to prior treatment with a CDK4/6 inhibitor alone or in combination with another SOC agent for breast cancer other than a CDK7 inhibitor. In some embodiments, the compound of Formula (I), (Ia), a species thereof, or a pharmaceutically acceptable salt thereof is co-administered with another SOC agent, such as an aromatase inhibitor (e.g. anastrozole, exemestane, or letrozole) or a SERM or SERD such as tamoxifen or fulvestrant, or a second line treatment after failure on an aromatase inhibitor or fulvestrant. In some embodiments, prior to administration of the compound of Formula (I), (Ia), a species thereof, or a pharmaceutically acceptable salt thereof, the patient is, has been determined to be, or has become resistant (after some initial responsiveness) to treatment with a CDK4/6 inhibitor alone or in combination with another SOC agent for breast cancer other than a CDK7 inhibitor, such as an aromatase inhibitor (e.g., anastrozole, exemestane, or letrozole), or a SERM or SERD such as tamoxifen or fulvestrant; and the compound of Formula (I), (Ia), a species thereof, or a pharmaceutically acceptable salt thereof is co-administered with a SOC agent for breast cancer (e.g., a second line treatment after failure of an aromatase inhibitor or a SERM or SERD such as tamoxifen or fulvestrant.

An enhancer or SE can be identified by various methods known in the art (see Hinsz et al., Cell, 155:934-947, 2013, McKeown et al., Cancer Discov., 7(10):1136-53, 2017; and U.S. Pat. Nos. 9181580 and 10,160,977, which are hereby incorporated herein by reference in their entireties). Identifying a SE can be achieved by obtaining a biological sample from a patient (e.g., from a biopsy or other source, as described herein). The important metrics for enhancer measurement occur in two dimensions: along the length of the DNA over which genomic markers (e.g., H3K27Ac) are contiguously detected and the compiled incidence of genomic marker at each base pair along that span of DNA, the compiled incidence constituting the magnitude. The measurement of the area under the curve (“AUC”) resulting from integration of length and magnitude analyses determines the strength of the enhancer. The strength of the KRAS SE relative to an appropriate reference can be used to diagnose (stratify) a patient and thereby determine whether a patient is likely to respond well to a compound of Formula (I), (Ia), a species thereof, or a pharmaceutically acceptable salt thereof. It will be readily apparent to one of ordinary skill in the art, particularly in view of the instant specification, that if the length of DNA over which the genomic markers is detected is the same for KRAS and the reference/control, then the ratio of the magnitude of the KRAS SE relative to the control will be equivalent to the strength and may also be used to determine whether a patient will be responsive to a compound of Formula (I), (Ia), a species thereof, or a pharmaceutically acceptable salt thereof. The strength of the KRAS SE in a cell can be normalized before comparing it to other samples. Normalization is achieved by comparison to a region in the same cell known to comprise a ubiquitous SE or enhancer that is present at similar levels in all cells. One example of such a ubiquitous super-enhancer region is the MALAT1 super-enhancer locus (chr11:65263724-65266724) (genome build hg19).

ChIP-seq is used to analyze protein interactions with DNA by combining chromatin immunoprecipitation (ChIP) with massively parallel DNA sequencing to identify the binding sites of DNA-associated proteins. It can be used to map global binding sites precisely for any protein of interest. Previously, ChIP-on-chip was the most common technique utilized to study these protein-DNA relations. Successful ChIP-seq is dependent on many factors including sonication strength and method, buffer compositions, antibody quality, and cell number (see, e.g., Furey, Nature Reviews Genetics 13:840-852, 2012); Metzker, Nature Reviews Genetics 11:31-46, 2010; and Park, Nature Reviews Genetics 10:669-680, 2009). Genomic markers other than H3K27Ac that can be used to identify SEs using ChIP-seq include P300, CBP, BRD2, BRD3, BRD4, components of the mediator complex (Loven et al., Cell, 153(2):320-334, 2013), histone 3 lysine 4 monomethylated (H3K4me1), and other tissue-specific enhancer tied transcription factors (Smith and Shilatifard, Nature Struct. Mol. Biol., 21(3):210-219, 2014; and Pott and Lieb, Nature Genetics, 47(1):8-12, 2015). Quantification of enhancer strength and identification of SEs can be determined using SE scores (McKeown et al., Cancer Discov. 7(10):1136-1153, 2017; DOI: 10.1158/2159-8290.CD-17-0399).

In some instances, H3K27Ac or other marker ChIP-seq data SE maps of the entire genome of a cell line or a patient sample already exist. One would then simply determine whether the strength, ordinal rank, or prevalence rank of the enhancer or SE in such maps at the chr8:128628088-128778308 (genome build hg19) locus was equal to or above the respective pre-determined threshold level. In some embodiments, one would simply determine whether the strength, ordinal rank, or prevalence rank of the enhancer or super-enhancer in such maps at the chr1:205399084-205515396 (genome build hg19) locus was equal to or above the respective pre-determined threshold level

The specific chromosomal location of KRAS and MALAT1 may differ for different genome builds and/or for different cell types. However, one of ordinary skill in the art, particularly in view of the instant specification, can determine such different locations by locating in such other genome builds specific sequences corresponding to the loci in genome build hg 19.

Other methods that can be used to identify SEs in the context of the present methods include chromatin immunoprecipitation (Delmore et al., Cell, 146(6):904-917, 2011), chip array (ChIP-chip), and chromatin immunoprecipitation followed by qPCR (ChIP-qPCR) using the same immunoprecipitated genomic markers and oligonucleotide sequences that hybridize to the chr8:128628088-128778308 (genome build hg19) MYC locus or chr1:205399084-205515396 (genome build hg19) CDK18 locus (for example). In the case of ChIP-chip, the signal is typically detected by intensity fluorescence resulting from hybridization of a probe and input assay sample as with other array-based technologies. For ChIP-qPCR, a dye that becomes fluorescent after intercalating the double stranded DNA generated in the PCR reaction is used to measure amplification of the template.

In some embodiments, determination of whether a cell has a KRAS SE strength equal to or above a requisite threshold level is achieved by comparing KRAS enhancer strength in a test cell to the corresponding KRAS strength in a population of cell samples, wherein each of the cell samples is obtained from a different source (e.g., a different patient, a different cell line, a different xenograft) reflecting the same disease to be treated. In some embodiments, only primary tumor cell samples from patients are used to determine the threshold level. In some aspects of these embodiments, at least some of the samples in the population will have been tested for responsiveness to a specific CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof) to establish: (a) the lowest KRAF enhancer strength of a sample in the population that responds to that specific compound (“lowest responder”); and, optionally, (b) the highest KRAF enhancer strength of a sample in the population that does not respond to that specific compound (“highest non-responder”). In these embodiments, a cutoff of KRAS enhancer strength above which a test cell would be considered responsive to that specific compound is set: i) equal to or up to 5% above the KRAS enhancer strength in the lowest responder in the population; or ii) equal to or up to 5% above the KRAS enhancer strength in the highest non-responder in the population; or iii) a value in between the KRAS enhancer strength of the lowest responder and the highest non-responder in the population.

In the above embodiments, not all of the samples in a population necessarily are to be tested for responsiveness to a specific CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof), but all samples are measured for KRAS enhancer strength. In some embodiments, the samples are rank ordered based on KRAS enhancer strength. The choice of which of the three methods set forth above to use to establish the cutoff will depend upon the difference in KRAS enhancer strength between the lowest responder and the highest non-responder in the population and whether the goal is to minimize the number of false positives or to minimize the chance of missing a potentially responsive sample or patient. When the difference between the lowest responder and highest non-responder is large (e.g., when there are many samples not tested for responsiveness that fall between the lowest responder and the highest non-responder in a rank ordering of KRAS enhancer strength), the cutoff is typically set equal to or is up to 5% above the KRAS enhancer strength in the lowest responder in the population. This cutoff maximizes the number of potential responders. When this difference is small (e.g., when there are few or no samples untested for responsiveness that fall between the lowest responder and the highest non-responder in a rank ordering of KRAS enhancer strength), the cutoff is typically set to a value in between the KRAS enhancer strength of the lowest responder and the highest non-responder. This cutoff minimizes the number of false positives. When the highest non-responder has a KRAS enhancer strength that is greater than the lowest responder, the cutoff is typically set to a value equal to or up to 5% above the KRAS enhancer strength in the highest non-responder in the population. This method also minimizes the number of false positives.

In some embodiments, the methods discussed above can be employed to simply determine if a diseased cell (e.g., a cancer cell) from a patient has a SE associated with a biomarker as described herein (e.g., KRAS). The presence of the SE indicates that the patient is likely to respond well to a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof). The cell is determined to have a SE associated with the biomarker (e.g., KRAS) when the enhancer has a strength that is equal to or above the enhancer associated with MALAT-1. In alternate embodiments, the cell is determined to have a SE associated with KRAS when the KRAS associated enhancer has a strength that is at least 10-fold greater than the median strength of all of the enhancers in the cell. In other embodiments, the cell is determined to have a SE associated with KRAS when the gene-associated enhancer has a strength that is above the point where the slope of the tangent is 1 in a rank-ordered graph of strength of each of the enhancers in the cell

In embodiments involving KRAS, the cutoff value for enhancer strength can be converted to a prevalence cutoff, which can then be applied to KRAS primary RNA transcript (e.g., pre-mRNA or mature mRNA) levels to determine an expression level cutoff value in a given assay for expression level.

In some embodiments, a feature of a genetic biomarker described herein (e.g., the presence of a mutation, a deletion, or primary RNA transcript levels (in, e.g., KRAS)) are used to determine sensitivity to a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (1), (Ia), a species thereof, or a pharmaceutically acceptable salt thereof) and thereby select patients for treatment.

In some embodiments, gene of interest/biomarker primary RNA transcript levels in a patient (as assessed, e.g., in a biological sample obtained from the patient) are compared, using the same assay, to the same gene of interest/biomarker primary RNA transcript levels in a population of patients having the same disease or condition to identify likely responders to a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof). Analogous comparisons can be made when another feature of the biomarker is selected for analysis (e.g., its copy number, chromosomal location, or expressed protein level). In embodiments where a biomarker (e.g., KRAS/K-ras) correlates with (e.g., is one whose expression correlates with) responsiveness to a compound of the invention, at least some of the samples in the population will have been tested for responsiveness to the CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof) to establish: (a) the lowest level (e.g., primary RNA transcript level) in a sample in the population that responds to that specific compound (“lowest RNA responder”), and, optionally, (b) the highest level (e.g., highest RNA level) in a sample in the population that does not respond to that specific compound (“highest RNA non-responder”). In these embodiments, a cutoff of biomarker primary RNA transcript level above which a test cell would be considered responsive to that specific compound is set: i) equal to or up to 5% above the level (e.g., the pre-mRNA or mature mRNA level) in the lowest RNA responder in the population (i.e., in the responder having the lowest expression of primary RNA transcripts); or ii) equal to or up to 5% above the level (e.g., the pre-mRNA or mature mRNA level) in the highest RNA non-responder in the population (i.e., in the non-responder having the highest level of expression of primary RNA transcripts); or iii) a value in between the level (e.g., RNA level) of the lowest responder and the highest non-responder in the population.

In embodiments where primary RNA (e.g., pre-mRNA or mature mRNA) transcript levels positively correlate with sensitivity to a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof), not all the samples in a population need to be tested for responsiveness to the CDK7 inhibitor but all samples are measured to determine the level of expression of the gene of interest (e.g., a primary RNA transcript (e.g., pre-mRNA or mature mRNA) level of KRAS or any other biomarker described herein). In some embodiments, the samples are rank ordered based on gene of interest primary RNA transcript levels (e.g., pre-mRNA or mature mRNA levels). The choice of which of the three methods set forth above to use to establish the pre-determined threshold or cutoff will depend upon the difference in gene of interest primary RNA transcript levels between the lowest RNA responder and the highest RNA non-responder in the population and whether the threshold or cutoff is designed to minimize false positives or maximize the potential number of responders. When this difference is large (e.g., when there are many samples not tested for responsiveness that fall between the lowest RNA responder and the highest RNA non-responder in a rank ordering of primary RNA transcript levels), the cutoff is typically set equal to or up to 5% above the RNA level in the lowest RNA responder. When this difference is small (e.g., when there are few or no samples untested for responsiveness that fall between the lowest RNA responder and the highest RNA non-responder in a rank ordering of primary RNA transcript levels), the cutoff is typically set to a value in between the RNA levels of the lowest RNA responder and the highest RNA non-responder. When the highest RNA non-responder has a primary RNA transcript level that is greater than the lowest RNA responder, the cutoff is typically set to a value equal to or up to 5% above the primary RNA transcript levels in the highest RNA non-responder in the population.

In embodiments where primary RNA transcript levels inversely correlate with sensitivity to a compound of the invention, not all of the samples in a population need to be tested for responsiveness to the compound, but all samples are measured for the gene of interest primary RNA transcript levels. In some embodiments, the samples are rank ordered based on gene of interest primary RNA transcript levels. The choice of which of the three methods set forth above to use to establish the cutoff will depend upon the difference in gene of interest primary RNA transcript levels between the highest RNA responder and the lowest RNA non-responder in the population and whether the cutoff is designed to minimize false positives or maximize the potential number of responders. When this difference is large (e.g., when there are many samples not tested for responsiveness that fall between the highest RNA responder and the lowest RNA non-responder in a rank ordering of primary RNA transcript levels), the cutoff is typically set equal to or up to 5% below the primary RNA transcript level in the highest primary RNA transcript responder. When this difference is small (e.g., when there are few or no samples untested for responsiveness that fall between the highest mRNA responder and the lowest mRNA non-responder in a rank ordering of primary RNA transcript levels), the cutoff is typically set to a value in between the RNA levels of the highest RNA responder and the lowest RNA non-responder. When the highest RNA responder has a primary RNA transcript level that is lower than the lowest primary RNA transcript responder, the cutoff is typically set to a value equal to or up to 5% below the RNA levels in the lowest RNA non-responder in the population.

In some aspects of embodiments where a test cell or sample is compared to a population, the cutoff primary RNA transcript level value(s) obtained for the population is converted to a prevalence rank and the primary RNA transcript level cutoff is expressed as a percent of the population having the cutoff value or higher, e.g., a prevalence cutoff.

Without being bound by theory, the Applicant believes that the prevalence rank of a test sample and the prevalence cutoff in a population will be similar regardless of the methodology used (to, for example, determine primary RNA transcript levels).

A patient can be identified as likely to respond well to a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof) if the state of KRAS as determined by, e.g., primary RNA transcript levels (e.g., pre-mRNA or mature mRNA levels) in a biological sample from the patient) corresponds to (e.g., is equal to or greater than) a prevalence rank in a population of about 80%, 79%, 78%, 77%, 76%, 75%, 74%, 73%, 72%, 71%, 70%, 69%, 68%, 67%, 66%, 65%, 64%, 63%, 62%, 61%, 60%, 59°,%, 58%, 57%, 56°,%, 55%, 54%, 43%, 42%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, or 20% as determined by the state of KRAS determined by assessing the same parameter (e.g., mature mRNA level(s)) in the population).

In still other embodiments, a population may be divided into three groups: responders, partial responders and non-responders, and two cutoff values (or thresholds) or prevalence cutoffs are set or determined. The partial responder group may include responders and non-responders as well as those patients whose response to a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof) was not as high as the responder group. This type of stratification may be particularly useful when, in a population, the highest RNA non-responder has a primary RNA transcript level that is greater than that of the lowest RNA responder. In this scenario, for KRAS (for example), the cutoff level or prevalence cutoff between responders and partial responders is set equal to or up to 5% above the KRAS primary RNA transcript level of the highest KRAS primary RNA non-responder; and the cutoff level or prevalence cutoff between partial responders and non-responders is set equal to or up to 5% below the KRAS primary RNA transcript level of the lowest KRAS primary RNA transcript responder. This type of stratification may be useful when the highest RNA responder has a primary RNA transcript level that is lower than that of the lowest RNA non-responder. In this scenario, the cutoff level or prevalence cutoff between responders and partial responders is set equal to or up to 5% below the primary RNA transcript level of the lowest primary RNA transcript level non-responder; and the cutoff level or prevalence cutoff between partial responders and non-responders is set equal to or up to 5% above the primary RNA transcript level of the highest primary RNA transcript responder. The determination of whether partial responders should be administered a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof) will depend upon the judgment of the treating physician and/or approval by a regulatory agency.

Methods that can be used to quantify specific RNA sequences (including the primary RNA transcripts or a biomarker described herein) in a biological sample are known in the art and include, but are not limited to, fluorescent hybridization such as utilized in services and products provided by NanoString Technologies, array based technology (Affymetrix), reverse transcriptase qPCR as with SYBR® Green (Life Technologies) or TaqMan® technology (Life Technologies), RNA sequencing (e.g., RNA-seq), RNA hybridization and signal amplification as utilized with RNAscope® (Advanced Cell Diagnostics), or Northern blot. In some cases, mRNA expression values for various genes in various cell types are publicly available (see, e.g., broadinstitute.org/ccle; and Barretina et al., Nature, 483:603-607, 2012). As noted, and if desired, one can analyze a cDNA molecule that is synthesized or reverse transcribed from a primary RNA transcript in lieu of analyzing the RNA transcript itself.

In some embodiments, the state of a biomarker (as assessed, for example, by the level of primary RNA transcripts) in both the test biological sample and the reference standard or all members of a population is normalized before comparison. Normalization involves adjusting the determined level of a primary RNA transcript by comparison to either another primary RNA transcript that is native to and present at equivalent levels in both of the cells (e.g., GADPH mRNA, 18S RNA), or to a fixed level of exogenous RNA that is “spiked” into samples of each of the cells prior to super-enhancer strength determination (Loven et al., Cell, 151(3):476-82, 2012; Kanno et al., BMC Genomics 7:64, 2006; Van de Peppel et al., EMBO Rep., 4:387-93, 2003).

A patient (e.g., a human) suffering from a cancer described herein and identified as described herein based on biomarker status may have been determined to be resistant (or to be acquiring resistance after some initial efficacy) to a therapeutic agent that was administered prior to the CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof). The therapeutic agent may have been a previously administered anti-cancer agent (e.g., a Bcl-2 inhibitor such as venetoclax, a BET inhibitor, a CDK4/6 inhibitor such as palbociclib or ribociclib, a CDK9 inhibitor such as alvocidib, a FLT3 inhibitor, a MEK inhibitor such a trametinib, a PARP inhibitor, such as olaparib or niraparib, a PI3K inhibitor, such as alpelisib or capecitabine, a platinum-based therapeutic agent such as cisplatin, oxaliplatin, nedaplatin, carboplatin, phenanthriplatin, picoplatin, satraplatin (JM216), or triplatin tetranitrate, a SERM, such as tamoxifen faloxifene, or toremifene, or a steroid receptor degrading agent (e.g., a SERD, such as fulvestrant). Combination therapies including one or more of these agents are also within the scope of the invention and are discussed further herein. For example, in one embodiment, the methods encompass the use of or administration of a CDK7 inhibitor, such as a compound of Formula (I), (Ia), a species thereof or a pharmaceutically acceptable salt thereof, in combination with a SERD, such as fulvestrant, to treat a cancer (e.g., a breast cancer (e.g., an ER⁺ breast cancer)) resistant to treatment with a CDK4/6 inhibitor such as palbociclib or ribociclib. In another embodiment, the methods encompass the use of or administration of a CDK7 inhibitor, such as a compound of Formula (I), (Ia), a species thereof or a pharmaceutically acceptable salt thereof, in combination with FOLFOX (folinic acid, fluorouracil, and oxaliplatin), FOLFIRI (folinic acid, fluorouracil, and irinotecan), or FOLFIRINOX (folinic acid, fluorouracil, irinotecan, and oxaliplatin) to treat, for example, a colorectal or pancreatic cancer.

In some embodiments, the prior therapeutic agent may be a platinum-based anti-cancer agent administered as a monotherapy or in combination with a SOC agent. Most cancer patients eventually develop resistance to platinum-based therapies by one or more of the following mechanisms: (i) molecular alterations in cell membrane transport proteins decrease uptake of the platinum agent; (ii) molecular alterations in apoptotic signaling pathways that prevent a cell from inducing cell death; (iii) molecular alterations of certain genes (e.g. BRCA½, CHEK1, CHEK2, RAD51) that restore the ability of the cell to repair platinum agent-induced DNA damage. Yamamoto el al., 2014, PloS ONE 9(8):el 05724. The term “molecular alterations” includes increased or decreased primary RNA transcript expression from the genes involved in these functions; increased or decreased expression of protein from such genes; and mutations in the RNA/proteins expressed from those genes.

Resistance is typically determined by disease progression (e.g., an increase in tumor size and/or numbers) during treatment or a decrease in the rate of shrinkage of a tumor. In some instances, a patient will be considered to have become resistant to a platinum-based agent when the patient’s cancer responds or stabilizes while on treatment, but which progresses within 1-6 months following treatment with the agent. Resistance can occur after any number of treatments with platinum agents. In some instances, disease progression occurs during, or within 1 month of completing treatment. In this case, the patient is considered to have never demonstrated a response to the agent. This is also referred to a being “refractory” to the treatment. Resistance may also be determined by a treating physician when the platinum agent is no longer considered to be an effective treatment for the cancer.

In some embodiments, the patient is or has been determined to be resistant to treatment with a CDK4/6 inhibitor administered as a monotherapy or in combination with a SOC agent.

Unlike platinum-based agents which are typically administered for a period of time followed by a period without treatment, CDK4/6 inhibitors, such as palbociclib, ribociclib or abemaciclib, are administered until disease progression is observed. In some instances, a patient will be considered to have become resistant to a CDK4/6 inhibitor when the patient’s cancer initially responds or stabilizes while on treatment, but which ultimately begins to progress while still on treatment. In some instances, a patient will be considered to be resistant (or refractory) to treatment with a CDK4/6 inhibitor if the cancer progresses during treatment without demonstrating any significant response or stabilization. Resistance may also be determined by a treating physician when the CDK4/6 inhibitor is no longer considered to be an effective treatment for the cancer.

The methods of the present invention can employ pharmaceutical compositions that include a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (la), a species thereof, or a pharmaceutically acceptable salt thereof) and, optionally, a pharmaceutically acceptable carrier. In certain embodiments, the pharmaceutical composition includes a compound of Formula (I) or a pharmaceutically acceptable salt thereof; a compound of Formula (Ia) or a pharmaceutically acceptable salt thereof; or a species of Formula (I) or (Ia) or a pharmaceutically acceptable salt thereof. As noted, a pharmaceutical composition can include one or more pharmaceutically acceptable carriers, and the active agent/ingredient (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof) can be provided therein in an effective amount (e.g., a therapeutically effective amount or a prophylactically effective amount).

Pharmaceutical compositions of the invention can be prepared by relevant methods known in the art of pharmacology. In general, such preparatory methods include the steps of bringing a compound described herein, including THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof, into association with a carrier and/or one or more other active ingredients (e.g., a second agent described herein) and/or accessory ingredients, and then, if necessary and/or desirable, shaping and/or packaging the product into a desired single-dose or multi-dose unit (e.g., for oral dosing). The accessory ingredient may improve the bioavailability of the CDK7 inhibitor (e.g., as just listed), may reduce and/or modify its metabolism, may inhibit its excretion, and/or may modify its distribution within the body (e.g., by targeting a diseased tissue (e.g., a tumor). The pharmaceutical compositions can be packaged in various ways, including in bulk containers and as single unit doses (containing, e.g., discrete, predetermined amounts of the active agent) or a plurality thereof, and any such packaged or divided dosage forms are within the scope of the invention. The amount of the active ingredient can be equal to the amount constituting a unit dosage or a convenient fraction of a dosage such as, for example, one-half or one-third of a dose.

Relative amounts of the active agent/ingredient, the pharmaceutically acceptable carrier(s), and/or any additional ingredients in a pharmaceutical composition of the invention can vary, depending upon the identity, size, and/or condition of the subject treated and further depending upon the route by which the composition is to be administered and the disease to be treated. By way of example, the composition may comprise between about 0.1% and 99.9% (w/w or w/v) of an active agent/ingredient.

Pharmaceutically acceptable carriers useful in the manufacture of the pharmaceutical compositions described herein are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable carriers useful in the manufacture of the pharmaceutical compositions described herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Pharmaceutical compositions used as described herein may be administered orally. Such orally acceptable dosage forms may be solid (e.g., a capsule, tablet, sachet, powder, granule, and orally dispersible film) or liquid (e.g., an ampoule, semi-solid, syrup, suspension, or solution (e.g., aqueous suspensions or dispersions and solutions). In the case of tablets, carriers commonly used include lactose and corn starch. Lubricating agents, such as magnesium stearate, can also be included. In the case of capsules, useful diluents include lactose and dried cornstarch. When aqueous suspensions are formulated, the active agent/ingredient can be combined with emulsifying and suspending agents. In any oral formulation, sweetening, flavoring or coloring agents may also be added. In any of the various embodiments described herein, an oral formulation can be formulated for immediate release or sustained/delayed release and may be coated or uncoated. A provided composition can also be micro-encapsulated.

Compositions suitable for buccal or sublingual administration include tablets, lozenges and pastilles. Formulations can also be prepared for subcutaneous, intravenous, intramuscular, intraocular, intravitreal, intra-articular, intra-synovial, intrasternal, intrathecal, intrahepatic, intraperitoneal intralesional and by intracranial injection or infusion techniques. Preferably, the compositions are administered orally, subcutaneously, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer’s solution and isotonic sodium chloride solution. In addition, sterile, fixed oils are conventionally employed as a solvent or suspending medium.

Although the descriptions of pharmaceutical compositions provided herein are principally directed to pharmaceutical compositions which are suitable for administration to humans, it will be understood by one of ordinary skill in the art that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification.

Compounds described herein are typically formulated in dosage unit form, e.g., single unit dosage form, for ease of administration and uniformity of dosage. The specific therapeutically or prophylactically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount can vary from subject to subject, depending, for example, on species, age, and general condition of a subject, severity of the side effects, disease to be treated, identity of the particular compound(s) to be administered, mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a CDK7 inhibitor for administration can be as known in the art. For example, a compound of Formula (I) or a pharmaceutically acceptable salt thereof can be administered one or more times a day (e.g., once) to a 70 kg adult human may comprise about 0.1-100 mg, about 1-100 mg, about 1-50 mg, about 1-35 mg (e.g., about 1-5, 1-10, 1-15, 1-20, 1-25, or 1-30 mg), about 2-20 mg, about 3-15 mg or about 10-30 mg (e.g., 10-20 or 10-25 mg). Here, and wherever ranges are referenced, the end points are included. The dosages provided in this disclosure can be scaled for patients of differing weights or body surface and may be expressed per m² of the patient’s body surface.

In certain embodiments, a compound of Formula (I) or a pharmaceutically acceptable salt thereof may be administered once per day. The dosage of a compound of Formula (I), (Ia), a species thereof or a pharmaceutically acceptable salt thereof (e.g., a salt thereof) can be about 0.1-100 mg, about 1-100 mg, about 1-50 mg, about 1-25 mg, about 2-20 mg, about 5-15 mg, about 10-15 mg, or about 13-14 mg.

In certain embodiments, a compound of Formula (I) may be administered twice per day. In some embodiments, the dosage of a compound of Formula I or a subgenus or species thereof for each administration is about 0.5 mg to about 50 mg, about 0.5 mg to about 25 mg, about 0.5 mg to about 1 mg, about 1 mg to about 10 mg, about 1 mg to about 5 mg, about 3 mg to about 5 mg, or about 4 mg to about 5 mg. THZ1 can be administered at a dose of about 10 mg/kg (e.g., intravenously, once or twice per day). ICE9042 can be administered at a dose of about 50 mg/kg to about 100 mg/kg, once or twice per day Alvocidib can be administered at a dose of about 1 mg/kg to about 10 mg/kg, once or twice per day, orally or parenterally (e.g., intravenously). SNS-32 can be administered at a dose of about 22 mg/m² parenterally (e.g., intravenously). SY-1365 can be administered at a dose of about 50 (e.g., 53) to about 80 mg/m² parenterally (e.g., intravenously over a period of about two hours). LY3405105 can be administered orally. Seliciclib can be administered at a dose of about 100 mg to about 800 mg (e.g., orally, BID).

In one embodiment, a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof) is administered in combination with second anti-cancer agent described herein or a plurality thereof. In one embodiment, the second anti-cancer agent is trametinib, optionally administered at a dosage of about 0.5 to about 5 mg daily or every other day. In another embodiment, the second anti-cancer agent is docetaxel, optionally administered at a dosage of about 20 mg to about 175 mg. In another embodiment, the second anti-cancer agent is gemcitabine, optionally administered at a dosage of about 1000 mg/m² intravenously every 4^{th} week on day 1, 8 and 15 or at a dosage of about 1250 mg/m² every 3^{rd} week on day 1 and 8 administered intravenously.

A CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof) or other composition described herein (e.g., a pharmaceutical composition) can be administered in a combination therapy (e.g., as defined and further described herein) with a second agent described herein (including those described as standard-of-care or to which a patient’s cancer may have become refractory) or a plurality thereof. The additional/second agent employed in a combination therapy is most likely to achieve a desired effect for the same disorder (e.g., the same cancer), however it may achieve different effects that aid the patient. Accordingly, the invention features pharmaceutical compositions containing a CDK7 inhibitor, such as a compound of Formula (1), (Ia), a species thereof, or a pharmaceutically acceptable salt thereof, in a therapeutically effect amount; a second agent selected from a Bcl-2 inhibitor such as venetoclax, a PARP inhibitor such as olaparib or niraparib, a platinum-based anti-cancer agent such as carboplatin, cisplatin, or oxaliplatin, a taxane such as paclitaxel, a CDK4/6 inhibitor such as palbociclib, ribociclib, abemaciclib, or trilaciclib, a selective estrogen receptor modulator (SERM) such as tamoxifen (available under the brand names Nolvadex™ and Soltamox™), raloxifene (available under the brand name Evista™), and toremifene (available as Fareston™) and a selective estrogen receptor degrader such as fulvestrant (available as Faslodex™), each in a therapeutically effective amount; and a pharmaceutically acceptable carrier. Kits containing such combinations of anti-cancer agents in separate containers are also within the scope of the present invention.

Unless otherwise specified, when employing a combination of a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (Ia), a species thereof, or a pharmaceutically acceptable salt thereof) and a second therapeutic agent in a therapeutic method, the second therapeutic agent can be administered concurrently with, prior to, or subsequent to the CDK7 inhibitor (e.g., a compound of Formula (I), (Ia), or a species thereof) or a pharmaceutically acceptable salt thereof. The second therapeutic pharmaceutical agent may be administered at a dose and/or on a time schedule determined for that pharmaceutical agent. The second therapeutic agent may also be administered together with the CDK7 inhibitor (e.g., a compound of Formula (I), (Ia), or a species thereof) or a pharmaceutically acceptable salt thereof in a single dosage form or administered separately in different dosage forms. In general, it is expected that the second therapeutic agents utilized in combination with a CDK7 inhibitor (e.g., a compound of Formula (I), (la), or a species thereof) or a pharmaceutically acceptable salt thereof will be utilized at levels that do not exceed the levels at which they are utilized individually. In some embodiments, the levels of the second therapeutic agent utilized in combination will be lower than those utilized in a monotherapy due to synergistic effects.

For combinations of a CDK7 inhibitor (e.g., THZ1, THZ2, SY-1365, YKL-5-124, ICEC0942, LY3405105, LDC4297, BS-181, alvocidib, seliciclib, SNS-32 or a pharmaceutically acceptable salt thereof, or a compound of structural Formula (I), (la), a species thereof, or a pharmaceutically acceptable salt thereof) and an additional/second agent selected from any one of those described herein, a kit comprising each of the two active therapeutics (or more, e.g., further including a third agent) can be provided and is within the scope of the present invention. Such kits find utility in any of the diagnostic and treatment methods described herein. In some instances, the first and second agents will be in separate vessels (e.g., with the first agent confined to a first container and the second agent confined to a second container) and/or formulated in a pharmaceutically acceptable composition, optionally in unit dosage form, that includes the first agent, the second agent, and a pharmaceutically acceptable carrier. In some instances, the kits include a written insert or label with instructions to use the two (or more) therapeutic agents in a patient suffering from a cancer (e.g., as described herein) and identified as amenable to treatment by a method described herein. The instructions may be adhered or otherwise attached to a vessel or vessels comprising the therapeutic agents. Alternatively, the instructions and the vessel(s) can be separate from one another but present together in a single kit, package, box, bag, or other type of container. The instructions in the kit will typically be mandated or recommended by a governmental agency approving the therapeutic use of the combination (e.g., in a patient population identified as described herein). The instructions may optionally comprise dosing information for each therapeutic agent, the types of cancer for which treatment of the combination was approved or may be prescribed, physicochemical information about each of the therapeutics, pharmacokinetic information about each of the therapeutics, drug-drug interaction information, or diagnostic information (e.g., based on a biomarker or a method of identifying a patient for treatment as described herein). The kits of the invention can also include reagents useful in the diagnostic methods described herein.

### EXAMPLES

The compounds described herein can be prepared from readily available starting materials and according to synthetic protocols known in the art and modifications thereof (see the reference materials described above; e.g., compounds of Formula (I) can be synthesized as described in WO 2020/093011 and U.S. Pat. No. 10,738,067). For example, it will be appreciated that where process conditions (e.g., reaction temperatures and times, mole ratios of reactants, solvents, pressures, etc.) are given, other process conditions can also be used. In addition, and as one of ordinary skill in the art will know, protecting groups may be used to prevent certain functional groups from undergoing undesired reactions. The choice of a suitable protecting group for a particular functional group as well as suitable conditions for protection and deprotection are well known in the art. For example, numerous protecting groups and guidance for their introduction and removal are disclosed by Greene et al. (Protecting Groups in Organic Synthesis, Second Edition, Wiley, New York, 1991, and references cited therein)

### Example 1: Tumor Growth Inhibition in PDX Models of CRC

We formulated Compound 101 by mixing it, in powder form, with 5% CAPTISOL® (a polyanionic beta-cyclodextrin derivative with a sodium sulfonate salt separated from the lipophilic cavity by a butyl ether spacer group, or sulfobutylether (SBE)). The mixture was formulated each day it was used and stored at 4° C. in the dark until ready for evaluation in CRC PDX models; we used 30 independent CRC PDX models. Ten of these tumors had mutations in the BRAF gene (models ST1207, ST428, ST540, ST2161, ST2148, ST1053, T1975, ST1163, and ST1419); ten had mutations in the KRAS gene (ST865, ST1660B, ST230, ST046, ST491C, ST1354, ST1192, ST094, ST238, and ST042); and ten had neither a BRAF nor KRAS mutation (ST2957, ST2168, ST1728, ST2838, ST2781, ST1756B, ST289, ST1996B, ST555B, and ST125). Each model was generated by implanting tumor fragments (~70 mm³) subcutaneously into multiple athymic nude mice. When tumors reached the appropriate range (150-300 mm³), animals were assigned to treatment and control groups and dosing was initiated (day 0): vehicle (n=3) versus Compound 101 (n=3). Compound 101 was administered by oral gavage at 6 mg/kg QD for 21 days followed by 1 week of observation.

For each PDX model, % tumor growth inhibition (%TGI) and % tumor regression (%TR) were calculated at the end of treatment (EoT, day 21) as:

$$\begin{matrix}\% \text{TGI} = \\ \left({\text{TV}}_{\text{Vehicle EoT}} - {\text{TV}}_{\text{Compound 1 EoT}}\right) / \left({\text{TV}}_{\text{Vehicle EoT}} - {\text{TV}}_{\text{All day 1}}\right) * 100\end{matrix}$$

$$\% \text{TR} = \left(\left({\text{TV}}_{\text{All day 1}} - {\text{TV}}_{\text{Compound 1 EoT}}\right) / {\text{TV}}_{\text{All day 1}}\right) * 100$$

“TV” is tumor volume, and “EoT” is end-of-treatment. “All day 1” indicates the average tumor volume in all animals on the first day of the study; the starting TV. The same formulas were used to calculate %TGI and %TR at the end of study when applicable.

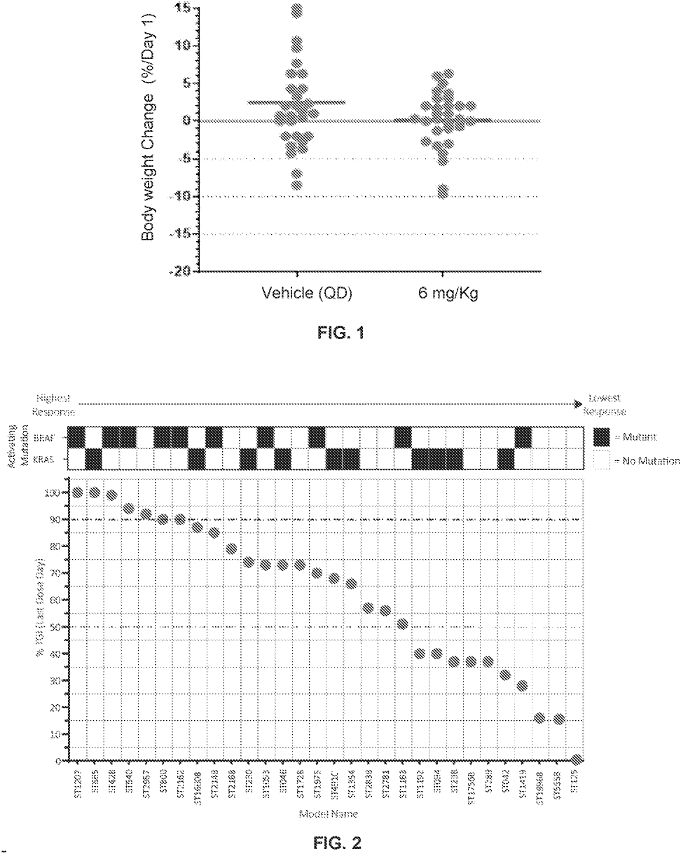

Compound 101 was well tolerated, with an average body weight change of 0% (-10% to +6%) at EoT (Day 21) across all 30 models (FIG. 1). Body weight loss, when observed, was reversed after treatment was discontinued. No treatment related deaths were observed on study.

Regarding anti-tumor activity, Compound 101 induced ≥50% TGI at end of treatment in 67% (20/30) of models (FIG. 2). Deep responses (≥90% TGI or regression) were observed in 23% (7/30) of models, with enrichment for deep responses in BRAF mutant models (50%, 5/10) relative to KRAS mutant (10%, ⅒), and wild-type (10%, ⅒) models. Of the seven models with deep responses, clear tumor regrowth was not observed in any model for 7 days after treatment discontinuation (day 28, end of study).

### Example 2: Studies of Compound 101, Trametinib, and Binimetinib

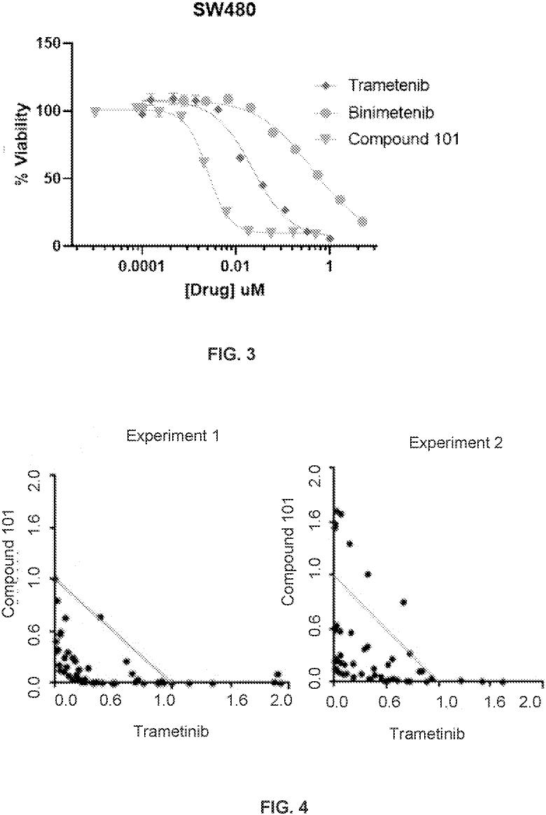

In the study described here, Compound 101 was more potent than the MEK inhibitors trametinib and binimetinib when applied to the KRAS mutant cell line SW480 (G12V) at various doses (see FIG. 3). Compound 101 (IC50 = 3 nM), trametinib (IC50 = 24 nM), and binimetinib (IC50 = 664 nM) induced a cytostatic response after five days of treatment (see. FIG. 3; Compound 101 (downward triangles; demonstrating the most growth inhibition), trametinib (diamonds) and binimetinib (circles; demonstating the least growth inhibition). When Compound 101 was administered to cells of the same cell line in combination with trametinib, we observed a synergistic effect (FIG. 4; n=2).

### Example 3: Analysis of Mutational Features in BRAF and KRAS PDX Models

Input data: We conducted a PDX study using 30 PDX models of CRC: 10 BRAF mutant models, 10 KRAS mutant models, and 10 BRAF/KRAS wildtype models. We treated each with Compound 101 at 6 mg/kg QD and with a vehicle control, with 3 replicate mice per condition. One replicate per condition was collected for downstream DNA and RNA analysis. DNA analysis was completed with whole exome sequencing, and RNA analysis was bulk polyA capture mRNA sequencing. Six mouse models were subsequently removed from the analysis due to contamination by mouse tissue or because they were deemed to be poor CRC model representations by comparison of their RNA-seq data with The Cancer Genome Atlas (TCGA) CRC RNA-seq data.

Feature Selection: We selected a set of 103 mutational features from the DNA results for further analysis. The selection was performed by first subsetting to DNA alterations predicted to have a strong functional impact, either by creating coding changes in genes or by creating a large deletion or amplification of a subset of a chromosome. These mutations were then subset to those that occur frequently in CRC (> 5% frequency in TCGA CRC data), and those with enough representation in our dataset to discover strong associations with outcome (occurring in 3-20 PDX models). Additionally, we filtered out copy number alterations that contained only unexpressed genes. All features were then binarized by presence or absence.

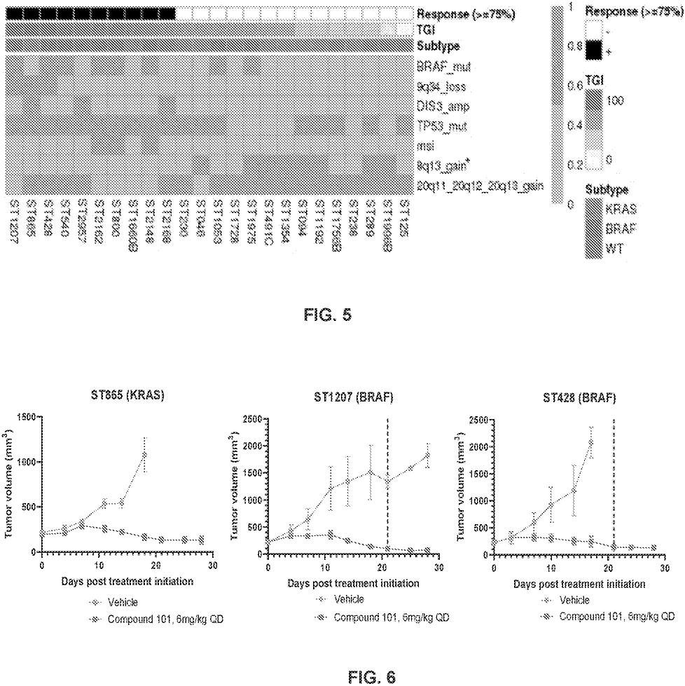

Elastic Net Model: These 103 features were then used to predict a response outcome in the PDX models, defined as TGI > 75%, using an elastic net regularized regression model (Zhou & Hastie, “Regularization and Variable Selection via the Elastic Nel”, Journal of the Royal Statistical Society, 2006), with parameter α=0.5. The elastic net constructs a parsimonious model of only the most relevant features and gives them a positive or negative weight based on their association with outcome. Finally, each selected feature was further evaluated by a fisher’s exact test with outcome. Seven features were selected and are displayed in FIG. 5, along with their presence (red (darker gray in the gray-scaled version)) or absence (grey (lighter gray in the gray-scaled version)) in the 30 PDX models, along with response (first two rows) and subtype (third row). Of the seven selected, BRAF mutations were the most positively predictive of response, while 9q34 heterozygous deletions, in particular, identified the three PDXs with complete tumor regressions (FIG. 6).

### Example 4: Tumor Growth Inhibition in PDX Models of PDAC

We formulated Compound 101 by mixing it, in powder form, with 5% CAPTISOL® (see above). We tested the formulation in eight independent PDAC PDX models. Seven of these tumors had a mutation in the KRAS gene (models ST1300, ST2478, ST390, ST1250, ST587, ST2426, ST569), and one had a mutation in the NRAS gene (ST1933). Animals were assigned to treatment and control groups and dosing was initiated (day 0): vehicle (n=3) versus Compound 101 (n=3). Compound 101 was administered by oral gavage at 6 mg/kg QD for 28 days.

For each PDX model, % tumor growth inhibition (%TGI) and % tumor regression (%TR; other abbreviations as above) were calculated at the end of treatment as:

$$\begin{matrix}\% \text{TGI} = \\ \left({\text{TV}}_{\text{Vehicle EoT}} - {\text{TV}}_{\text{compound 101 EoT}}\right) / \left({\text{TV}}_{\text{Vehicle EoT}} - {\text{TV}}_{\text{All day 1}}\right) \\ * 100\end{matrix}$$

$$\% \text{TR} = \left(\left({\text{TV}}_{\text{All day 1}} - {\text{TV}}_{\text{compound 101}}\right) / {\text{TV}}_{\text{All day 1}}\right) * 100$$

The same formulas were used to calculate %TGI and %TR at end of study when applicable.

Compound 101 was well tolerated, with an average body weight change of 0% (-4% to +5%) across all eight models. No treatment-related deaths were observed on study. Regarding anti-tumor activity, Compound 101 induced ≥50% TGI at end of treatment in 75% (6/8) of the models. Regressions were observed in 50% (4/8) of the models (FIG. 8). Two models (ST1300 and ST 2478) were observed for two weeks post-dosing, and we found that regressions were sustained during this period.

### Example 5: Studies of Compound 101 and Gemcitabine

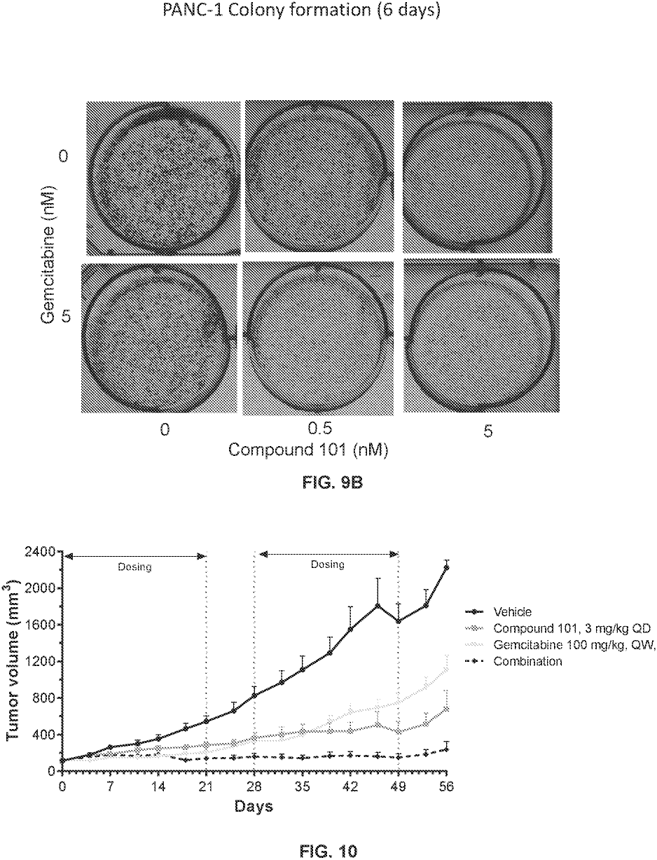

When Compound 101 was administered to PANC-1 cells in combination with gemcitabine, we observed an enhanced effect. Cell growth was assayed, and the results are summarized in the line graphs of FIG. 9A. The cultured cells were also stained with crystal violet, which stains nuclei a deep purple color and thereby aids visualization (FIG. 9B).

### Example 6: Tumor Growth Inhibition in CDX Models of PDAC

We formulated Compound 101 by mixing it, in powder form, with 5% CAPTISOL® (see above). We used the PDAC CDX model PANC-1 (KRAS G12D; see Lieber et al., Intl. J. Cancer, 15(5):741-747, 1975). Each mouse was inoculated subcutaneously at the right flank with 5×10⁶ PANC-1 cells in 0.2 ml of base media for tumor development. Animals were assigned to treatment and control groups and dosing was initiated (day 0): vehicle (n=5) versus Compound 101 (n=5). Compound 101 was administered by oral gavage at 3 mg/kg QD. A separate cohort of mice were dosed with gemcitabine, ip, at 100 mg/kg QW. A third cohort were dosed with a combination of 3 mg/kg QD Compound 101 and 100 mg/kg gemcitabine ip QW. All mice were dosed for 21 days followed by one week of observation and then a second 21-day cycle of dosing.

For each PDX model, % tumor growth inhibition (%TGI) and % tumor regression (%TR; other abbreviations as above) were calculated at the end of treatment (EoT, day 21) as:

$$\begin{matrix}\% \text{TGI} = \\ \left({\text{TV}}_{\text{Vehicle EoT}} - {\text{TV}}_{\text{Compound 101 EoT}}\right) / \left({\text{TV}}_{\text{Vehicle EoT}} - {\text{TV}}_{\text{All day 1}}\right) \\ * 100\end{matrix}$$

$$\% \text{TR} = \left(\left({\text{TV}}_{\text{All day 1}} - {\text{TV}}_{\text{Compound 101 EoT}}\right) / {\text{TV}}_{\text{All day 1}}\right) * 100$$

The same formulas were used to calculate %TGI at the end of study.

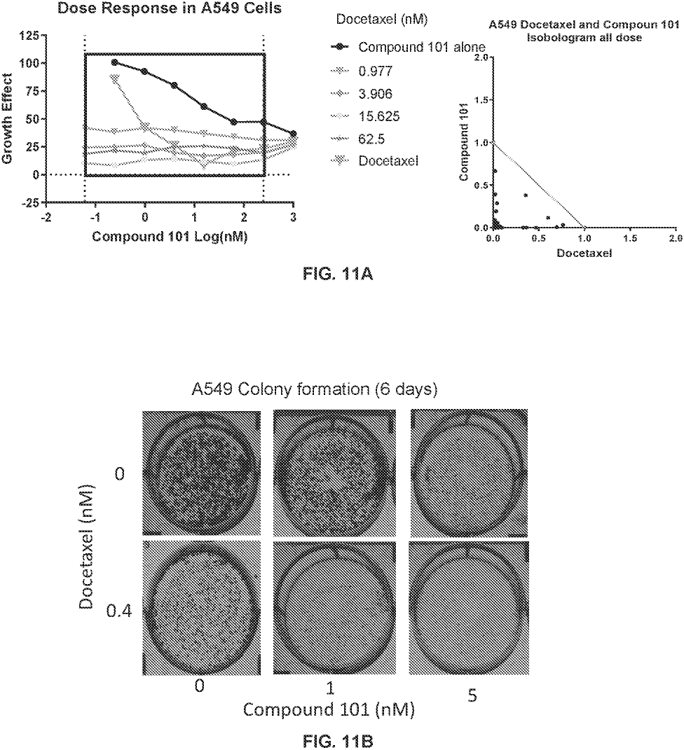

Compound 101 was well tolerated, with an average body weight change of 0% (-avg-BWC ±2%). Regarding anti-tumor activity. Compound 101 induced 72% TGI at end of treatment and the combination of Compound 101 and gemcitabine induced 97% TGI (FIG. 10). Similar combination results (94.3% TGI) were seen with a Compound 101 dosing regimen of 3 mg/kg QD administered every other week for 28 days.

### Example 7: Studies of Compound 101 and Docetaxel

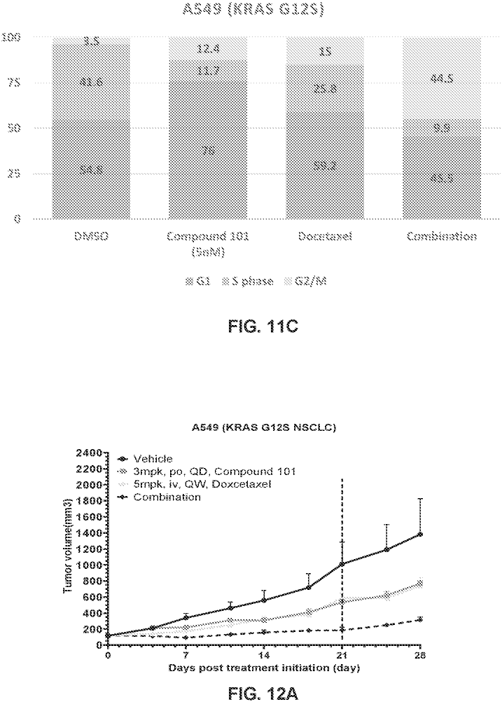

When Compound 101 was administered to A549 cells in combination with docetaxel, we observed an enhanced effect (FIGS. 11A-11C).

### Example 8: Tumor Growth Inhibition in Models of NSCLC

We formulated Compound 101 by mixing it, in powder form, with 5% CAPTISOL® (see above). We used NSCLC PDX model ST2972 (KRAS G12C) or NSCLC CDX model A549 (KRAS G12S). Compound 101 was administered by oral gavage at 3 mg/kg QD. Separate cohorts of A549 mice were dosed with docetaxel. iv. 5 mg/kg QW. A separate cohort of ST2972 mice were dosed with docetaxel, iv, 10 mg/kg once per week. Mice were dosed with a combination of docetaxel and Compound 101. All mice were dosed for 21 days followed by one week of observation.

For each xenograft model, % tumor growth inhibition (%TGI) and % tumor regression (%TR; other abbreviations as above) were calculated at the end of treatment as:

$$\begin{matrix}\% \text{TGI} = \\ \left({\text{TV}}_{\text{Vehicle EoT}} - {\text{TV}}_{\text{Compound 101 EoT}}\right) / \left({\text{TV}}_{\text{Vehicle EoT}} - {\text{TV}}_{\text{All day 1}}\right) \\ * 100\end{matrix}$$

$$\% \text{TR} = \left(\left({\text{TV}}_{\text{All day 1}} - {\text{TV}}_{\text{Compound 101 EoT}}\right) / {\text{TV}}_{\text{All day 1}}\right) * 100$$

The same formulas were used to calculate %TGI at the end of study.

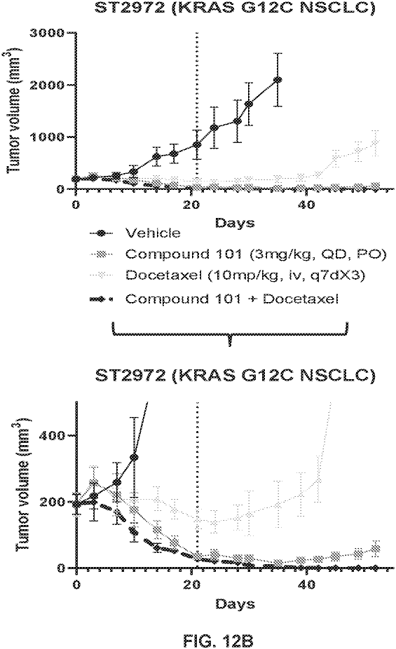

In A549 mice, Compound 101 induced 53% TGI and in combination with docetaxel (5 mg/kg QW) induced 87.9% TGI (FIGS. 12A-12B). In ST2972 tumors, Compound 101 induced near complete regressions, and in combination with docetaxel (10 mg/kg QW) induced complete regressions with no tumor regrowth for ≥ 4 weeks post drug discontinuation. Both regimens were well-tolerated (avg-BWC +3.6% to -6%)

### Example 9: Studies of Compound 101, BI-3406, and Trametinib

We formulated Compound 101 by mixing it, in powder form, with 5% CAPTISOL® (see above). For this study, we employed the PDAC CDX model PANC-1 (KRAS G12D). Each mouse was inoculated subcutaneously at the right flank with 5×10⁶ PANC-1 cells in 0.2 ml of base media for tumor development. Animals were assigned to treatment and control groups and dosing was initiated (Day 0): vehicle (n=5) versus Compound 101 (n=5). Compound 101 was administered by oral gavage at 3 mg/kg QD. An additional cohort of mice were dosed with trametinib at 0.5 mg/kg QD. Yet another cohort of mice were dosed with BI-3406 (Son of Sevenless 1 inhibitor) at 50 mg/kg BID. Other cohorts of mice were dosed with combinations of the therapeutic agents. All mice were dosed for 21 days; observed for one week, subjected to a second cycle of dosing for 21 days; and then observed for an additional one week.

For each PDX model, % tumor growth inhibition (%TGI) and % tumor regression (%TR; other abbreviations as above) were calculated at the end of treatment as:

$$\begin{matrix}\% \text{TGI} = \\ \left({\text{TV}}_{\text{Vehicle EoT}} - {\text{TV}}_{\text{Compound 101 EoT}}\right) / \left({\text{TV}}_{\text{Vehicle EoT}} - {\text{TV}}_{\text{All day 1}}\right) \\ * 100\end{matrix}$$

$$\% \text{TR} = \left(\left({\text{TV}}_{\text{All day 1}} - {\text{TV}}_{\text{Compound 101 EoT}}\right) / {\text{TV}}_{\text{All day 1}}\right) * 100$$

The same formulas were used to calculate %TGI at the end of study.

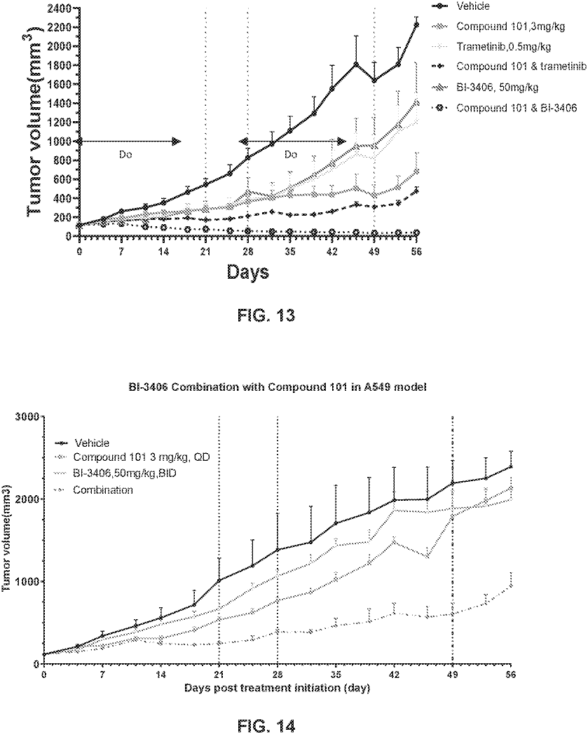

Compound 101 was well tolerated, with an average body weight change of 0% (-avg-BWC +2%). Regarding anti-tumor activity, Compound 101 induced 77% TGI at end of treatment, and the treatment with a combination of Compound 101 and trametinib indued 87% TGI (FIG. 13). The Compound 101 and BI-3406 combination treatment induced regressions (FIGS. 13-14).

### Example 10: Study of Compound 101 on 24 Ovarian and Breast Cancer Cell Lines

An in vitro cancer cell line screen was performed on 24 ovarian and breast cancer cell lines treated with concentrations of Compound 101 ranging from 30 pM to 500 nM. After 5 days of treatment, cell lines were assayed using a CellTiter-Glo Luminescent Cell Viability Assay (Promega). Fluorescence values of Compound 101 treated cells were compared to those of vehicle control treated cells taken at both end of treatment and start of treatment. Growth rate metrics were calculated using the following formulas:

$${\text{rate}}_{\text{c}} = log \text{} 2 \left(\text{control} {\text{day}}_{\text{5}} / {\text{day}}_{\text{0}}\right)$$

$${\text{rate}}_{\text{t}} = log \text{} 2 \left(\text{treated} {\text{day}}_{\text{5}} / {\text{day}}_{\text{0}}\right)$$

$$\text{GR} = 2 \hat{} \left({\text{rate}}_{\text{t}} / {\text{rate}}_{\text{c}}\right) - 1$$

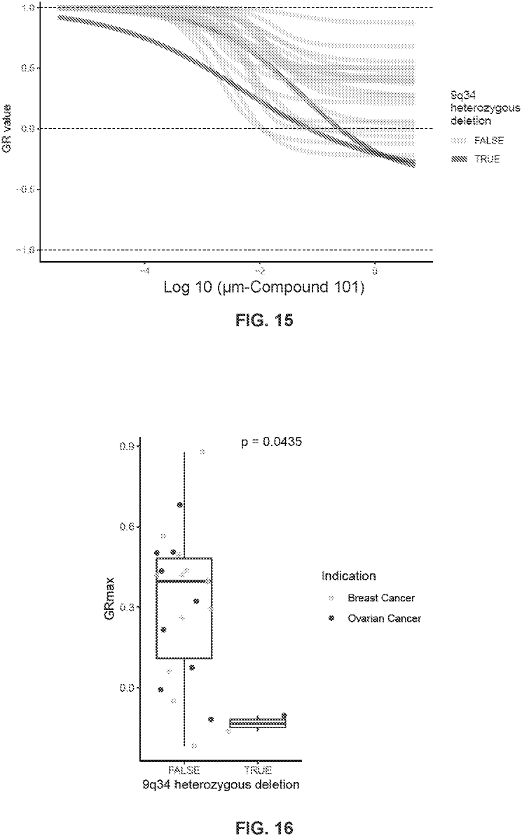

Here, “control day₅” refers to the fluorescence value of the vehicle control measured at day 5, “treated days” refers to the fluorescence value of compound 101 treated cells at day 5, and “day₀” refers to the fluorescence value of cells at the start of treatment. GR values were then used to fit a 3-parameter logistic regression, from which GRmax was estimated using 500 nM compound 101 (FIG. 15).

Copy-number data from the Cancer Cell Line Encyclopedia was used to calculate the copy number of the 9q34 cyotgenetic band in the abovementioned cell lines. A Mann-Whitney U-test was then used to compare the GRmax values of 9q34 heterozygously-deleted cell lines to those not heterozygously-deleted (FIG. 16).

The invention encompasses all variations, combinations, and permutations in which one or more limitations, elements, clauses, and descriptive terms from one or more of the listed claims are introduced into another claim. For example, any claim that is dependent on another claim can be modified to include one or more limitations found in any other claim that is dependent on the same base claim. Where elements are presented as lists, e.g., in Markush group format, every possible subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

1. A method of treating cancer in a selected patient, the method comprising administering a therapeutically effective amount of a CDK7 inhibitor to the patient, wherein the patient has been determined to have a cancer in which
(a) a KRAS gene is mutated, is genetically amplified, contains an epigenetic alteration, is translocated, is transcribed at a level equal to or above a pre-determined threshold, or encodes a protein that is mutant, translated at a level equal to or above a pre-determined threshold, or has increased activity relative to a reference standard; and/or
(b) chromosomal band 9q34 is completely or partially deleted.

2. The method of claim 1, wherein the CDK7 inhibitor is THZ1, THZ2, SY-1365, YKL-5-124, ICEC0942, LY3405105, LDC4297, BS-181, alvocidib, seliciclib, SNS-32, or a compound of structural Formula (I): 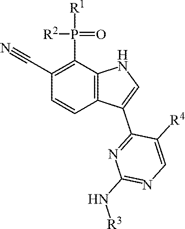
or a pharmaceutically acceptable salt of any of the foregoing, optionally within a pharmaceutical composition, wherein:
R¹ is methyl or ethyl;
R² is methyl or ethyl;
R³ is 5-methylpiperidin-3-yl, 5,5-dimethylpiperidin-3-yl, 6-methylpiperdin-3-yl, or 6,6-dimethylpiperidin-3-yl, wherein one or more hydrogen atoms in R³ is optionally replaced by deuterium; and
R⁴ is —CF₃ or chloro.

3. The method of claim 2, wherein the compound conforms to Formula (I) and (i) R¹ is methyl and R² is methyl or (ii) R¹ is methyl and R² is ethyl.

4. The method of claim 3, wherein R⁴ is —CF₃.

5. The method of claim 3, wherein R⁴ is chloro.

6. The method of claim 3, wherein R³ is 5-methylpiperidin-3-yl or 6-methylpiperdin-3-yl.

7. The method of claim 3, wherein R³ is 5,5-dimethylpiperidin-3-yl or 6,6-dimethylpiperidin-3-yl.

8-9. (canceled)

10. The method of claim 3, wherein the compound has structural Formula (Ia): 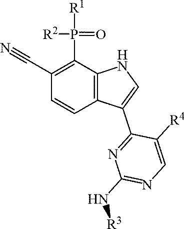
or the pharmaceutically acceptable salt thereof, wherein R³ is 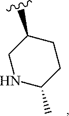 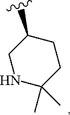 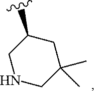 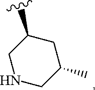 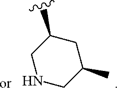
.

11-13. (canceled)

14. The method of claim 10, wherein the compound is: 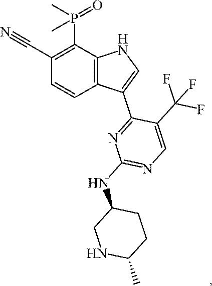 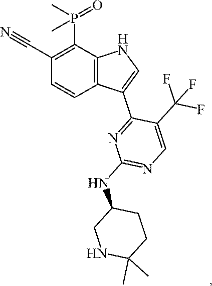 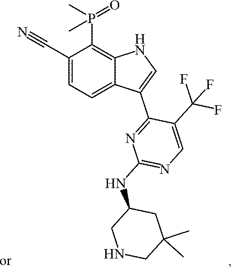
or is a pharmaceutically acceptable salt of any one of the foregoing compounds.

15. The method of claim 14, wherein the compound is 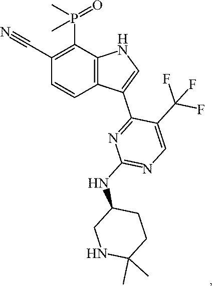
or a pharmaceutically acceptable salt thereof.

16. (canceled)

17. The method of claim 1, wherein the patient has been determined to have a cancer in which a KRAS gene is mutated, is genetically amplified, contains an epigenetic alteration, is translocated, is transcribed at a level equal to or above a pre-determined threshold, or encodes a protein that is mutant, translated at a level equal to or above a pre-determined threshold, or has increased activity relative to a reference standard; and in which one or more of the following, additional biomarkers have been determined to be positive: BCL2L1, BRAF, DIS3, WNT, 1p36, msi, 8q and 20q.

18. The method of claim 17, wherein DIS3 is has been determined to be positive by virtue of amplification-dependent overexpression, chromosomal band 1p36 has been determined to be positive by virtue of deletion, or 8q has been determined to be positive by virtue of amplification or gain of function.

19. The method of claim 1, wherein the patient has been determined to have a cancer in which chromosomal band 9q34 is completely or partially deleted and in which one or more of the following, additional biomarkers have been determined to be positive: BCL2L1, BRAF, DIS3, WNT, 1p36, msi, 8q, and 20q.

20. The method of claim 19, wherein BRAF has been determined to be positive.

21. The method of claim 1, wherein the cancer is a colorectal cancer, lung cancer, optionally NSCLC, pancreatic cancer, optionally PDAC, breast cancer, cancer of a reproductive organ, optionally ovarian cancer, bile duct, the skin, bladder, liver, kidney, or bone or wherein the cancer comprises cells in which CDK7 is overexpressed, misexpressed, or overactive relative to expression or activity in a reference standard.

22. The method of claim 21, wherein the patient has been determined to have a cancer in which a KRAS gene is mutated, is genetically amplified, contains an epigenetic alteration, is translocated, is transcribed at a level equal to or above a pre-determined threshold, or encodes a protein that is mutant, translated at a level equal to or above a pre-determined threshold, or has increased activity relative to a reference standard and the cancer is a colorectal cancer, lung cancer, or pancreatic cancer.

23. The method of claim 21, wherein the patient has been determined to have a cancer in which chromosomal band 9q34 is completely or partially deleted and the cancer is a cancer affecting a reproductive organ, bile duct, the skin, bladder, liver, lung, kidney, or bone.

24. The method of claim 1, wherein the CDK7 inhibitor constitutes a first anti-cancer agent and the patient has undergone, is presently undergoing, or is prescribed treatment with a second anti-cancer agent.

25. The method of claim 24, wherein the second anti-cancer agent is a Bcl-2 inhibitor; a hormone receptor degradation agent; a Flt3 (FMS-like tyrosine kinase 3) inhibitor; a PARP inhibitor; a BET inhibitor; a platinum-based therapeutic agent; a CDK4/6 inhibitor; a MEK inhibitor; or a phosphoinositide 3-kinase (PI3 kinase) inhibitor.

26. The method of claim 24, wherein the Bcl-2 inhibitor is APG-1252, APG-2575, BP1002 (prexigebersen), the antisense oligonucleotide known as oblimersen (G3139), S55746/BCL201, or venetoclax; the CDK9 inhibitor is alvocidib/DSP-2033/flavopiridol, AT7519, AZD5576, BAY1251152, BAY1143572, CYC065, nanoflavopiridol, NVP2, seliciclib (CYC202), TG02, TP-1287, VS2-370, or voruciclib (formerly P1446A-05); the hormone receptor degradation agent is fulvestrant; the Flt3 inhibitor is CDX-301, CG′806, CT053PTSA, crenolanib, ENMD-2076, FF-10101-01, FLYSYN, gilteritinib (ASP2215), HM43239, lestautinib, ponatinib, NMS-088, sorafenib, sunitinib, pacritinib, pexidartinib/PLX3397, quizartinib, midostaurin, SEL24, SKI-G-801, or SKLB1028; the PARP inhibitor is olaparib, rucaparib, talazoparib, veliparib (ABT-888), or niraparib; the BET inhibitor is ABBV-075, BAY-299, BAY-1238097, BMS-986158, CPI-0610, CPI-203, FT-1101, GS-5829, GSK-2820151, GSK-525762, I-BET151, I-BET762, INCB054329, JQ1, MS436, OTX015, PFI-1, PLX51107, RVX2135, TEN-010, ZEN-3694, or a compound disclosed in U.S Application No. 12/810,564 (now U.S. Pat. No. 8,476,260); the platinum-based therapeutic agent is cisplatin, oxaliplatin, nedaplatin, carboplatin, phenanthriplatin, picoplatin, satraplatin (JM216), or triplatin tetranitrate; the CDK4/6 inhibitor is BPI-1178, G1T38, palbociclib, ribociclib, ON 123300, trilaciclib, or abemaciclib; the MEK inhibitor is trametinib; and the phosphoinositide 3-kinase (PI3 kinase) inhibitor is idelalisib, copanlisib, duvelisib, or alpelisib; or capecitabine.
